(19) United States
(12) Patent Application Publication
(10) Pub. No.: US 2003/0072716 A1
(43) Pub. Date: Apr. 17, 2003

(54) Renewable, carbohydrate based CO2-philes

(76) Inventors: Raveendran Poovathinthodiyil, Durham, NC (US); Scott L. Wallen, Chapel Hill, NC (US)

Correspondence Address: JENKINS & WILSON, PA, 3100 TOWER BLVD, SUITE 1400, DURHAM, NC, 27707, US

(21) Appl. No.: 10/177,564

(22) Filed: Jun. 21, 2002

Related U.S. Application Data
(60) Provisional application No. 60/300,219, filed on Jun. 22, 2001.

(51) Int. Cl. A61K 9/00; A61K 31/70; A61L 9/04; A61K 31/715

(52) U.S. Cl. 424/43; 514/23; 514/53; 514/61

(57) ABSTRACT

A composition is disclosed comprising a carbohydrate-based material a dispersed in carbon dioxide. A general method for synthesizing inexpensive, renewable, non-toxic, non-fluorous, carbohydrate based CO₂-philes is disclosed. These CO₂-philes are soluble in carbon dioxide. Methods of making the composition are also disclosed. The methods and composition are useful in a variety of applications and can utilize gaseous, liquid and supercritical carbon dioxide. The methods and compositions are useful in the synthesis of surfactants and metal chelates for CO₂, as a sizing substrate, in CO₂-based coating processes, for impregnation and plasticizing cellulosic and non-cellulosic materials, in pharmaceutical applications, such as crystallization, dispersion and encapsulation of bioactive molecules in solid systems, in the densification of CO₂, in the synthesis of biodegradable polymers in CO₂, and for carbon dioxide removal, to name just a few.

### CROSS REFERENCE TO RELATED APPLICATIONS

The present patent application is based on and claims priority to U.S. Provisional Application Serial No. 60/300,219, entitled “RENEWABLE, CARBOHYDRATE BASED CO₂-PHILES”, which was filed Jun. 22, 2001 and is incorporated herein by reference.

### TECHNICAL FIELD

The present invention generally relates to CO₂-philic materials, and compositions comprising carbohydrates and carbohydrate-based materials adapted to interact with carbon dioxide in gaseous, liquid and supercritical forms. The invention also relates to methods of producing the same and applications in which the compositions and the CO₂-philic moieties can be employed.

Abbreviations

| | |
|---|---|
| AGLU | alpha 1,2,3,4,6-pentaacetyl-D-glucose |
| AIBN | 2,2′-azobisisobutyronitrile |
| BGLU | beta 1,2,3,4,6-pentaacetyl-D-glucose |
| BGLA | beta 1,2,3,4,6-pentaacetyl β-D-galactose |
| CO₂ | carbon dioxide |
| DFT | density functional theory |
| EOR | enhanced oil recovery |
| GAS | gas anti-solvent |
| HOMO | highest occupied molecular orbital |
| SCO₂ | supercritical carbon dioxide |
| LA | Lewis acid |
| LB | Lewis base |
| RESS | Rapid Expansion of Supercritical Solutions |
| scCO2 | supercritical carbon dioxide |

### BACKGROUND ART

Carbon dioxide, e.g., liquid and supercritical carbon dioxide (scCO₂), is playing an increasingly significant role as a successful green replacement solvent(s) for organic liquids. Carbon dioxide offers economical and environmental benefits, due to its favorable physical and chemical properties. Recyclability, non-toxicity, ease of solvent removal, and readily tunable solvent parameters make CO₂ a desirable potential alternative over many conventional solvents. The relatively low solubility of polar and non-volatile compounds in scCO₂, however, has been a sizable drawback and thus potentially limits the application of CO₂ in a number of chemical and industrial processes.

Some attempts to enhance the solubility of certain molecules in carbon dioxide, a molecule of interest have involved derivatizing the molecule of interest, particularly with fluoro groups. Molecular systems derivatized with fluorocarbon groups have been recognized to increase the solubility of compounds in CO₂ by several orders of magnitude. Fluorocarbon-based CO₂-philes are expensive, however, and moreover, recent studies suggest that the degradation products of fluorocarbon polymers can potentially have a negative impact on the environment. Thus, although perfluoro- and siloxane systems show increased solubility in CO₂, their potentially high cost could limit widespread use of these materials as CO₂-philes for various processes in the CO₂ solvent system in future applications.

Hydrocarbons substituted with carbonyl groups have been proposed as economically viable, environmentally benign CO₂-philes. The high solubility of these carbonyl systems in scCO₂ was attributed to the Lewis acid (LA)-Lewis base (LB) interactions between CO₂ and CO₂-philic Lewis base functionalities such as carbonyl groups (Sarbu et al., (2000) Nature 405:165-168; Kazarian et al., (1996) J. Am. Chem. Soc. 118:1729-1736; Nelson & Borkman, (1998) J. Phys. Chem. A 102:7860-7863). Ab initio calculations (Nelson & Borkman, (1998) J. Phys. Chem. A 102:7860-7863) indicate that the interaction between the carbonyl groups of an acetate functionality and CO₂ is almost half as strong as the hydrogen bond interaction in a water dimer. IR spectroscopic studies (Kazarian et al., (1996) J. Am. Chem. Soc. 118:1729-1736) have confirmed this view of specific interactions between CO₂ and the carbonyl groups. Based on these revelations, by optimizing the enthalpic and entropic factors, Beckman and co-workers synthesized hydrocarbon based, carbonyl supported, poly-(ether-carbonate) copolymers soluble in liquid CO₂ by maximizing the entropic and enthalpic contributions to solvation (Sarbu et al., (2000) Nature 405:165-168). These investigators also reported a high solubitity for poly-(propylene glycol) acetate with 21 repeat units (Sarbu et al., (2000) Nature 405:165-168).

Thus, principles related to the design of CO₂-philic molecules, including amphiphiles, have attracted great interest, and different molecular level approaches have been employed to “CO₂-philize” compounds that are otherwise insoluble in CO₂ (DeSimone et al., (1992) Science 267: 945-947; Rindfleisch et al., (1996) J. Phys. Chem. 100: 15581-15587; Sarbu et al., (2000) Nature 405:165-168; Laintz et al., (1991) J. Supercrit. Fluids 4: 194-198).

Carbohydrates are renewable materials and there are efforts to synthesize novel and useful carbohydrate-based compounds. Such compounds are desirable, in view of their environmentally benign attributes, as compared to presently-available fluoro- and petroleum-based compounds. Prior to the disclosure of the present invention, however, researchers have been unable to form a composition comprising a carbohydrate-based material dispersed in carbon dioxide, either as gaseous CO₂, liquid CO₂ or supercritical CO₂. This is due, in part, to the fact that carbohydrate molecules typically comprise hydroxyl groups, making them CO₂-phobic and immiscible with CO₂.

Synthesis of inexpensive, non-toxic and CO₂-philic derivatives from carbohydrates is of interest to “green” chemistry. Further, a composition comprising a carbohydrate-based material dispersed in carbon dioxide, as well as methods of making and using the composition, would have a wide range of uses and would find application in the pharmaceutical industry, the oil industry, the textile industries, the paper and coating industry and the wood industry, to name just a few fields that would benefit from such a composition.

Accordingly, there is a need for a composition of matter comprising a carbohydrate-based material adapted to be dispersed in carbon dioxide, as well as a method of preparing the carbohydrate-based material. There is also a need for alternative, economically viable, renewable CO₂-philic materials having the ability to act as co-solvents and/or the ability to be modified to form a surfactant to dissolve polar and amphiphilic materials in CO₂. Also of importance is the synthesis of renewable materials that can absorb and/or adsorb CO₂. Such materials can be employed in operations involving CO₂ removal from a gas stream containing CO₂. The present invention solves these and other applications.

### SUMMARY OF THE INVENTION

A composition comprising a carbohydrate-based material dispersed in carbon dioxide is disclosed. The carbohydrate-based material comprises a carbohydrate and at least one non-fluorous CO₂-philic group.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of forming a composition comprising a carbohydrate-based material dispersed in carbon dioxide is disclosed. In a preferred embodiment, the method comprises: (a) providing a CO₂-phobic carbohydrate comprising one of one or more hydroxyl groups and one or more or ring hydrogens; (b) chemically replacing at least one of a hydroxyl group and a ring hydrogen with a non-fluorous CO₂-philic group to form a carbohydrate-based material; and (c) dispersing the carbohydrate-based material in carbon dioxide, whereby a composition comprising a carbohydrate-based material dispersed in carbon dioxide is formed.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of modulating the viscosity of a composition comprising carbon dioxide is disclosed. In a preferred embodiment, the method comprises: (a) providing a carbohydrate-based material adapted for dispersion in carbon dioxide, wherein the carbohydrate-based material comprises a carbohydrate and at least one non-fluorous CO₂-philic group; and (b) dispersing an amount of the carbohydrate-based material in a composition comprising carbon dioxide sufficient to modulate the viscosity of the composition comprising carbon dioxide to a desired viscosity.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of chelating a metal atom disposed in carbon dioxide is disclosed. In a preferred embodiment, the method comprises: (a) providing a CO₂-philic carbohydrate-based material comprising a carbohydrate, at least one non-fluorous CO₂-philic group and at least one chelating group covalently linked to one of the CO₂-philic group and the carbohydrate; and (b) contacting the carbohydrate-based material with a sample comprising carbon dioxide, in which a metal atom is known or suspected to be disposed.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of sizing a substrate is disclosed. In a preferred embodiment, the method comprises: (a) providing a carbohydrate-based material comprising a carbohydrate, at least one non-fluorous CO₂-philic group and at least one moiety known or suspected to be an effective size; (b) dispersing the carbohydrate-based material in carbon dioxide to form a sizing solution; and (c) contacting substrate with the sizing solution, whereby a substrate is sized.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of sorbing carbon dioxide from a sample is disclosed. In a preferred embodiment, the method comprises: (a) providing a CO₂-philic carbohydrate-based material comprising a carbohydrate and at least one non-fluorous CO₂-philic group; and (b) contacting the CO₂-philic carbohydrate-based material with a sample known or suspected to comprise carbon dioxide, whereby carbon dioxide is sorbed from a sample.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of isolating a carbohydrate ester from a sample is disclosed. In a preferred embodiment, the method comprises: (a) providing a sample known or suspected to comprise a carbohydrate ester; (b) contacting the sample with carbon dioxide to form an extraction mixture; and (c) isolating the extraction mixture from the sample, whereby a carbohydrate ester is isolated from a sample.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide.

A method of synthesizing a polymer is disclosed. In a preferred embodiment, the method comprises: (a) providing a carbohydrate-based material comprising a non-fluorous CO₂-philic group; (b) joining the carbohydrate-based material with a compound comprising a polymerizable group to form a seed unit; (c) dispersing the seed unit in carbon dioxide; and (d) initiating polymerization, whereby a polymer is synthesized.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of impregnating or plasticizing a matrix comprising a cellulosic or non-cellulosic material is disclosed. In a preferred embodiment, the method comprises: (a) providing a carbohydrate-based material comprising a carbohydrate, at least one non-fluorous CO₂-philic group and at least one moiety known or suspected to be an effective size; (b) dispersing the carbohydrate-based material in CO₂ to form a treatment solution; and (c) contacting a substrate to be impregnated or plasticized with the treatment solution, whereby a matrix comprising a cellulosic or non-cellulosic material is impregnated or plasticized.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of isolating a carbohydrate material from a CO₂ solution is disclosed. In a preferred embodiment, the method comprises: (a) providing a carbohydrate-based material comprising a carbohydrate and a non-fluorous CO₂-philic group; (b) dispersing the carbohydrate-based material in CO₂ to form a CO₂ solution; and (c) spraying the CO₂ solution through a nozzle.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙRₙ, where Rₙ and R_{n′} are independently hydrogen or an alkyl group.

A method of encapsulating a compound in a carbohydrate-based material is disclosed. In a preferred embodiment, the method comprises: (a) providing a carbohydrate-based material; (b) dispersing the carbohydrate-based material in CO₂ to form a CO₂ solution; and (c) dispersing the compound in the CO₂-solution, whereby a compound is encapsulated in a carbohydrate-based material.

A method of producing a carbohydrate-based mesoporous material is disclosed. In a preferred embodiment, the method comprises: (a) providing a carbohydrate-based material comprising a carbohydrate and a non-fluorous CO₂-philic group; (b) dispersing the carbohydrate-based material in CO₂ disposed in a pressurizable vessel to form a CO₂ solution; and (c) rapidly releasing the CO₂ solution from the vessel, whereby a carbohydrate-based mesoporous material is produced.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of crystallizing a carbohydrate-based material from a CO₂ solution is disclosed. In a preferred embodiment, the method comprises: (a) dispersing a carbohydrate-based material comprising a carbohydrate and a non-fluorous CO₂-philic group in a pressurizable vessel containing CO₂ to form a CO₂ solution; and (b) expanding the CO₂ solution by slow release of CO₂ from the vessel, whereby a carbohydrate-based material is crystallized.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

A method of producing a glassy and fibrous material from a carbohydrate-based material is disclosed. In a preferred embodiment, the method comprises: (a) melting a carbohydrate-based material comprising a carbohydrate and a non-fluorous CO₂-philic group with CO₂ to form a CO₂ melt; (b) contacting a crystal formation structure with the CO₂ melt; and (c) removing the crystal formation structure from the CO₂-melt, whereby a glassy and fibrous material is produced from a carbohydrate-based material.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of solubilizing a dye in carbon dioxide is disclosed. In a preferred embodiment, the method comprises:(a) providing a carbohydrate-based material comprising a carbohydrate and a non-fluorous CO₂-philic group, and a CO₂-phobic dye molecule; (b) chemically associating the carbohydrate based material with the CO₂-phobic dye molecule to form a CO₂-soluble dye molecule; and (c) dispersing the CO₂-soluble dye molecule in CO₂, whereby a dye is solubilized in carbon dioxide.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

A method of solubilizing a catalyst in CO₂ is disclosed. In a preferred embodiment, the method comprises: (a) providing a carbohydrate-based material comprising a carbohydrate and a non-fluorous CO₂-philic group and a catalyst molecule; (b) chemically associating the carbohydrate-based material and the catalyst molecule to form a CO₂ soluble catalyst; and (c) dispersing the CO₂ soluble catalyst in CO₂, whereby a catalyst is solubilized in CO₂.

Preferably, the carbon dioxide is in a form selected from the group consisting of supercritical carbon dioxide, liquid carbon dioxide and gaseous carbon dioxide. Preferably, the carbohydrate is selected from the group consisting of a monosaccharide, a disaccharide, a trisaccharide, a polysaccharide, a cyclic saccharide and an acyclic saccharide. Preferably, the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

A method of extracting a carbohydrate-containing molecule from a matrix using CO₂ is disclosed. In a preferred embodiment, the method comprises: (a) providing a matrix comprising a CO₂-phobic carbohydrate-containing molecule; (b) contacting the matrix with acetic anhydride and acetic acid to form an acetylated carbohydrate-containing molecule; (c) extracting the acetylated carbohydrate molecule from the matrix, using carbon dioxide as a solvent to form extracted carbohydrate molecules; and (d) hydrolyzing the extracted carbohydrate molecules, whereby a carbohydrate-containing molecule is extracted.

An object of the invention having been stated hereinabove, other objects will be evident as the description proceeds, when taken in connection with the accompanying Drawings and Laboratory Examples as best described hereinbelow.

### BRIEF DESCRIPTION OF THE DRAWINGS

FIG. 1 is a diagram depicting the highest occupied molecular orbital (HOMO) for the optimized geometry of the CO₂-methyl acetate complex calculated at the MP2/6-31+G* level. The C—H O hydrogen bond acts cooperatively with the Lewis acid-Lewis base interaction (CO₂-carbonyl) introducing further stabilization.

FIG. 2A is a cartoon depicting a ball-and-stick representation of an optimized structure of AGLU.

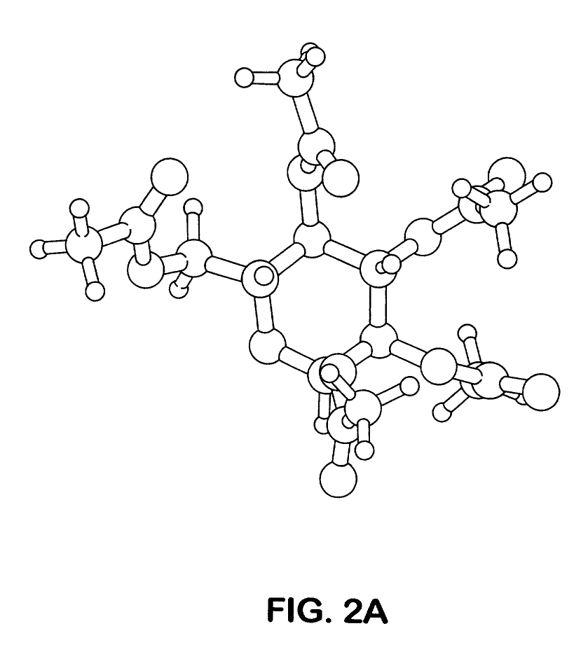

FIG. 2B is a cartoon depicting a ball-and-stick representation of an optimized structure of BGLU.

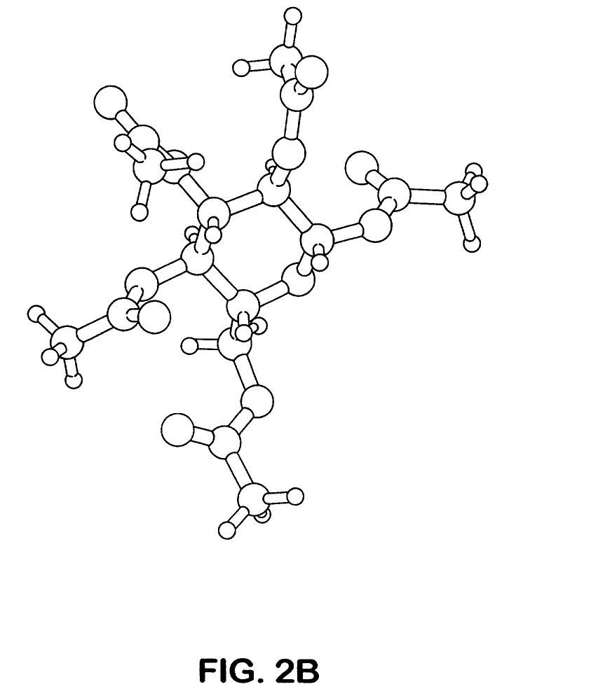

FIG. 2C is a cartoon depicting a ball-and-stick representation of an optimized structure of BGAL.

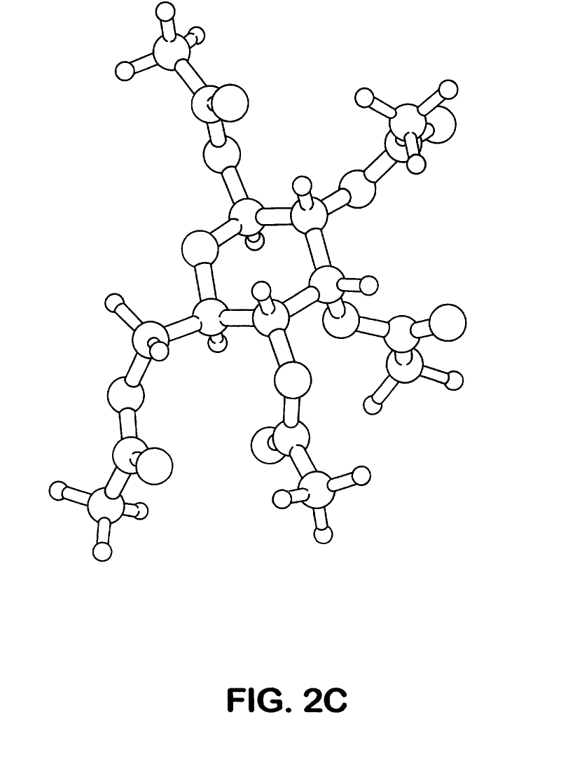

FIG. 3 is a photograph depicting the deliquescence, swelling, and dissolution of BGLU in CO₂ at 23.0° C.: Panel (A) depicts solid material; Panel (B) depicts the material at the deliquescence pressure (55.9 bar) with a gaseous CO₂ phase in contact with the viscous liquid BGLU forming the lower phase; Panel (C) depicts the swelling of the BGLU liquid phase with an increase of CO₂ pressure (57.9 bar); Panel (D) depicts the continued swelling of the BGLU liquid phase with an increase of CO₂ pressure (58.9 bar); Panel (E) depicts the melt phase at the CO₂ liquid-vapor equilibrium pressure (60.5 bar) and after stirring; and Panel (F) depicts complete miscibility of the melt in liquid CO₂ with additional CO₂ (60.5 bar).

FIG. 4 is plot depicting the cloud-point pressure versus the weight percentage of the carbohydrate derivative for AGLU ( ), BGLU ( ), and BGAL ( ) in supercritical CO₂ at a temperature of 40.0° C.

FIG. 5 is an OptiCam microscope image of a glassy fiber of β-cyclodectrin triacetate pulled from a CO₂-induced melt of a β-cyclodectrin triacetate sample.

FIG. 6A is a ball-and-stick figure depicting the crystal structure of BGAL in crystals grown from supercritical carbon dioxide solution at 40.0° C.

FIG. 6B is a ball-and-stick figure depicting the packing of BGAL in crystals grown from supercritical carbon dioxide solution at 40.0° C.

### DETAILED DESCRIPTION OF THE INVENTION

Liquid and supercritical carbon dioxide is regarded as an environmentally benign solvent due to its relative non-toxicity. It is also an excellent choice for use as a solvent, due to its ease of removal from a system, its abundance, its easily achieved critical parameters and liquid-vapor coexistence boundary, its low cost, and its tunability of solvent parameters. See, e.g., DeSimone et al., (1992) Science 267:945-947; Eckert et al., (1996) Nature 373:313-318; McHugh & Krukonis, Supercritical Fluid Extractions: Principles and Practice, 2^{nd} ed. Butterworth-Heinerman: Boston, Mass., (1994); Rindfleisch et al., (1996) J. Phys. Chem. 100:15581-15587.

The low solubility of the majority of non-polar, polar and ionic materials has, however, been a limitation in expanding the possibilities of this solvent system. See Consani & Smith, (1990) J. Supercrit. Fluids 3:51-65. Also, attempts to use conventional surfactants in CO₂ failed as a result of the poor solubility of these materials, despite their high solubility in non-polar solvents such as ethane and propane. Thus, the fundamental principles for the design of CO₂-soluble molecules, including amphiphiles, have attracted great interest, and different approaches have been made at the molecular level to “CO₂-philize” compounds that are otherwise insoluble in CO₂. The first, and presently the most widely applied, method is the introduction of fluorocarbons. For example, DeSimone and coworkers synthesized homo and copolymers of fluorinated acrylates that exhibit complete miscibility in CO₂ (see DeSimone et al., (1992) Science 267:945-947).

CO₂-phobic compounds (i.e. compounds that are not soluble in CO₂) can be made soluble in CO₂ by incorporating one or more CO₂-philic groups. Compounds that are soluble in CO₂ are of significant interest, in part, because CO₂-soluble materials can be employed in a number of chemical and industrial processes that employ CO₂ as a solvent, as well as processes that can be adapted to use CO₂ as a solvent. For example, one of ordinary skill in the art can synthesize CO₂-soluble surfactants, metal chelates and other types of compounds of interest by associating a CO₂-philic group with the a carbohydrate.

As noted above, a common approach to enhancing the solubility of a compound in CO₂ is by preparing a fluoro derivative of the compound. Indeed, prior to the present disclosure, the most CO₂-soluble compounds available are fluorinated hydrocarbons. For example, Johnston et al. synthesized a hybrid alkyl/fluoroalkyl surfactant and a perfluoropolyether surfactant that was soluble in CO₂ and which solubilized significant amounts of water (Johnston et al., (1996) Science 271: 624-626). However, fluorocarbons are expensive and make processes that employ these materials as CO₂-philes economically unfavorable. Thus, one of the challenges in the area of CO₂-based applications is to identify a method of preparing inexpensive, environmentally benign compounds that are soluble in CO₂, preferably from a renewable resource, and more preferably from carbohydrates. Moreover, these prior approaches do not address carbohydrates, a class of compounds that would be valuable in CO₂-based systems and applications, if they could be solubilized in that solvent.

Another challenge is the design of inexpensive CO₂-philic materials that are adapted to remove CO₂ from a gas stream comprising CO₂. Many of the CO₂-philes disclosed herein are adapted for this purpose, while others have a number of industrial applications and can be employed as, for example, a plasticizer, an insecticide, a bittering agent, and a soaker for paper. In these and other applications, CO₂ is preferably employed as a medium. In accordance with the present invention, these and other compounds can be designed, upon consideration of the present disclosure.

1. Definitions

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or ±10%, more preferably ±5%, even more preferably ±1%, and still more preferably ±0.1% from the specified amount, as such variations are appropriate to perform the disclosed method.

As used herein, the term “adsorb,” and grammatical derivatives thereof, means a surface phenomena wherein CO₂ becomes attached to the surface of the carbohydrate-based material by chemically interacting with the surface molecules (i.e., chemisorption). The “absorb” also refers to a bulk phenomena wherein the CO₂ diffuses into the inner structure of the carbohydrate-based material.

As used herein, the term “carbohydrate” means a compound comprising carbon atoms, hydrogen atoms and oxygen atoms. Representative carbohydrates that can be useful in the present invention include glucose and galactose. A carbohydrate (or a carbohydrate-based material) can comprise atoms in addition to carbon, hydrogen and oxygen, but will contain at least these types of atoms. The term “carbohydrate” encompasses both cyclized and open chain forms of a compound comprising carbon, hydrogen and oxygen; thus, compounds comprising open chains, such as sorbitol and mannitol, are also encompassed by the term “carbohydrate.”

As used herein, the term “carbohydrate-based material” means any compound comprising a carbohydrate and an additional chemical moiety, preferably a CO₂-philic group. More preferably the additional chemical group has been substituted for a group normally found on a carbohydrate, such as a hydyroxyl group, or even a ring hydrogen. An additional chemical moiety can comprise a functional group (e.g. an acetyl group) or even a single atom (e.g. an oxygen atom). Thus, a carbohydrate-based material specifically encompasses a carbohydrate comprising a CO₂-philic group.

As used herein, the terms “carbon dioxide” and “CO₂” are used interchangeably and mean a molecule comprising a carbon atom and two oxygen atoms. The term also encompasses molecules formed from isotopes of carbon and oxygen. Carbon dioxide can take several forms, including gaseous, liquid and supercritical, and unless otherwise indicated, the terms “carbon dioxide” and “CO₂” encompass all forms of carbon dioxide.

As used herein the term “CO₂-phile,” and grammatical derivations thereof, means any chemical compound that can be dispersed in carbon dioxide, liquefied by CO₂, or that can undergo deliquescence upon contact with CO₂ (preferably gaseous CO₂). The term also refers to a chemical compound that can sorb (i.e. absorb or adsorb) carbon dioxide. There is no limitation on either the chemical compound or the form of carbon dioxide. Thus, a CO₂-phile can comprise a compound that can be dispersed in liquid carbon dioxide or supercritical carbon dioxide, or that can sorb gaseous carbon dioxide. Preferred CO₂-philes include chemically modified (e.g. acetylated or benzoylated) carbohydrates.

As used herein, the term “CO₂-philic group” means a chemical moiety, preferably a functional group, which, when associated with a target molecule or chemical moiety, modulates the solubility of the target molecule or chemical moiety in carbon dioxide in one or more of its forms, including liquid carbon dioxide or supercritical carbon dioxide, or facilitates the sorption of gaseous carbon dioxide on the target molecule. Preferred CO₂-philic groups include acetyl groups, benzoyl groups, phosphonyl groups and sulfonyl groups. A CO₂-philic group preferably comprises a Lewis base group.

As used herein, the terms “CO₂-phobe” and “CO₂-phobic” refer to a compound that is not soluble in supercritical or liquid CO₂. A CO₂-phobe or a CO₂-phobic material will also not interact with (e.g. sorb) gaseous carbon dioxide.

As used herein, the term “disperse” is used in its broadest sense and means dissolving or melting a material in another material, which can comprise a solvent. For example, a carbohydrate-based material of the present invention can be dispersed in carbon dioxide by dissolving it in liquid or supercritical carbon dioxide. A carbohydrate-based material can also be dispersed by contacting it with gaseous carbon dioxide, upon which it can melt. Thus melting and dissolving are processes that are encompassed by the term “disperse.” Dispersing can be achieved with or without agitation.

As used herein, the term “Lewis base” means a compound comprising a Lewis base group.

As used herein, the term “Lewis base group” means a functional group that is capable of partially or fully donating a lone pair of electrons to an electrophilic functionality (i.e. a Lewis acid), whereby an interactive stabilization by partial charge transfer is possible.

As used herein, the terms “liquid carbon dioxide” and “liquid CO₂” are used interchangeably to mean carbon dioxide in liquid form. Carbon dioxide takes a liquid form when subjected to a pressure of at least about 5.11 bar (corresponding to the triple point) in a temperature range between about 216.8 K (corresponding to the triple point) and about 304.2 K (corresponding to the critical point). Liquid carbon dioxide has a density between about 0.7 and about 1.2 g/ml and a viscosity of about 0.07 mN/m². Liquid carbon dioxide can be distinguished from other phases of carbon dioxide based on its surface tension, which is about 5 dynes/cm for liquid carbon dioxide.

As used herein, when referring to the treatment of a substrate with a compound, the term “size” means any material that is applied to the substrate. For example, in the textile industry a size refers to a material applied to yarn or other textile during the manufacturing process. In the paper manufacturing industry, a size refers to a material applied to paper during or after the paper is manufactured.

As used herein, the terms “supercritical carbon dioxide” and “supercritical CO₂” are used interchangeably and mean carbon dioxide under conditions of pressure and temperature that are above the critical pressure (P_{c}=about 71 atm) and temperature (T_{c}=about 31° C.). In this state, the CO₂ has approximately the viscosity of the corresponding gas and a density that is quantitatively intermediate between the density of the liquid and gas states. Both properties are tunable (i.e. controllably variable).

As used herein, the term “interact,” and grammatical derivatives thereof, means interactions between molecules, such as, for example, hydrogen bonding between two molecules, van der Waals interactions between two molecules and Lewis acid-Lewis base-type of interactions between two molecules. The interaction can be, but need not be, detectable.

As used herein, the term “soluble” means a property of a chemical species that refers to the ability of the chemical species to become dispersed in a solvent. In the context of the present invention, the term refers to the ability of a carbohydrate or carbohydrate-based material to be dispersed in carbon dioxide in the gaseous, liquid or supercritical state.

As used herein, the term “sorb” encompasses both absorption and adsorption and refers to a compound or the ability of a compound to non-covalently associate with another compound.

As used herein, the terms “supercritical” and “supercritical phase” refer to a condition when a substance, exceeds a critical temperature and pressure, at which point the material cannot be condensed into the liquid phase despite the addition of further pressure.

As used herein, the term “supercritical carbon dioxide” means carbon dioxide which is at or above the critical temperature of about 31° C. and the critical pressure of about 71 atmospheres and which cannot be condensed into a liquid phase despite the addition of further pressure. The thermodynamic properties of CO₂ are reported in Hyaft, (1984) J. Org. Chem. 49: 5097-5101, incorporated herein by reference.

II. General Considerations

The following sections present a brief discussion of several aspects of the present invention that are common to some embodiments of the invention disclosed herein.

11.A. Carbon Dioxide In one aspect of the present invention, carbon dioxide is employed as a solvent or a dispersion medium. In this role, carbon dioxide can be employed in a gaseous, liquid or supercritical phase. In one embodiment, a composition of the present invention employs carbon dioxide as a continuous phase, in the liquid or supercritical conditions, with a carbohydrate-based material being solubilized or dissolved therein as described herein. In the context of the present invention, a composition comprising a carbohydrate-based material dispersed in carbon dioxide preferably comprises from above about 0, 5, 10, 20, or 30 to about 70, 80, 90, 95, or 98 percent by weight of carbon dioxide.

Carbon dioxide in liquid form can be employed in some embodiments of the present invention. If liquid CO₂ is employed in the present invention, the temperature employed during a process involving liquid CO₂ is preferably below about 31° C., which is the critical temperature for carbon dioxide. Above about 31° C., carbon dioxide is in the supercritical phase and cannot be liquefied by the application of pressure.

In some embodiments of the present invention, CO₂ is employed in its supercritical phase. In general, the methods and syntheses disclosed in aspects of the present invention can be carried out under any temperature and pressure ranges, with a carbohydrate derivative employed under conditions in which carbon dioxide is in its gaseous, liquid or supercritical forms. In particular, the methods of the present invention are preferably carried out at a temperature range from about −100° C. to about 225° C. The pressures employed preferably range from about 15 psig to about 10,000 psig.

Carbon dioxide employed in the present invention can comprise additional components. Representative components that can co-exist with carbon dioxide, and can therefore be employed in the methods of the present invention, can include, but are not limited to, water, toughening agents, colorants, dyes, biological agents, food, pharmaceuticals, rheology modifiers, plasticizing agents, flame retardants, antibacterial agents, flame retardants, co-solvents, surfactants and co-surfactants.

II.B. Carbohydrates

In one aspect of the present invention, carbohydrate molecules are employed. In some aspects of the present invention, carbohydrate monomers are preferably employed. A carbohydrate monomer of the present invention, such as glucose, for example, comprises an aldehyde group (first carbon position) and five hydroxyl groups, whereas fructose contains a keto group (at second carbon position) and five hydroxyl groups. Many carbohydrate monomers form a five (furanoside) or six (pyranoside) member ring between the aldehyde or keto group and one of the hydroxyl groups at 4th or 5th carbon position of the molecule. A newly formed hydroxyl group (anomeric hydroxyl) at the original functional group has two isomers: alpha or beta anomer, depending on down or up of the hydroxyl position.

Various types of carbohydrates can be employed in the present invention, including small and large cyclic and acyclic carbohydrates. Preferred carbohydrates include, without limitation, monosaccharides, disaccharides, trisaccharides, and polysaccharides.

Some of the carbohydrates that can form a component of a carbohydrate-based material of the present invention include:

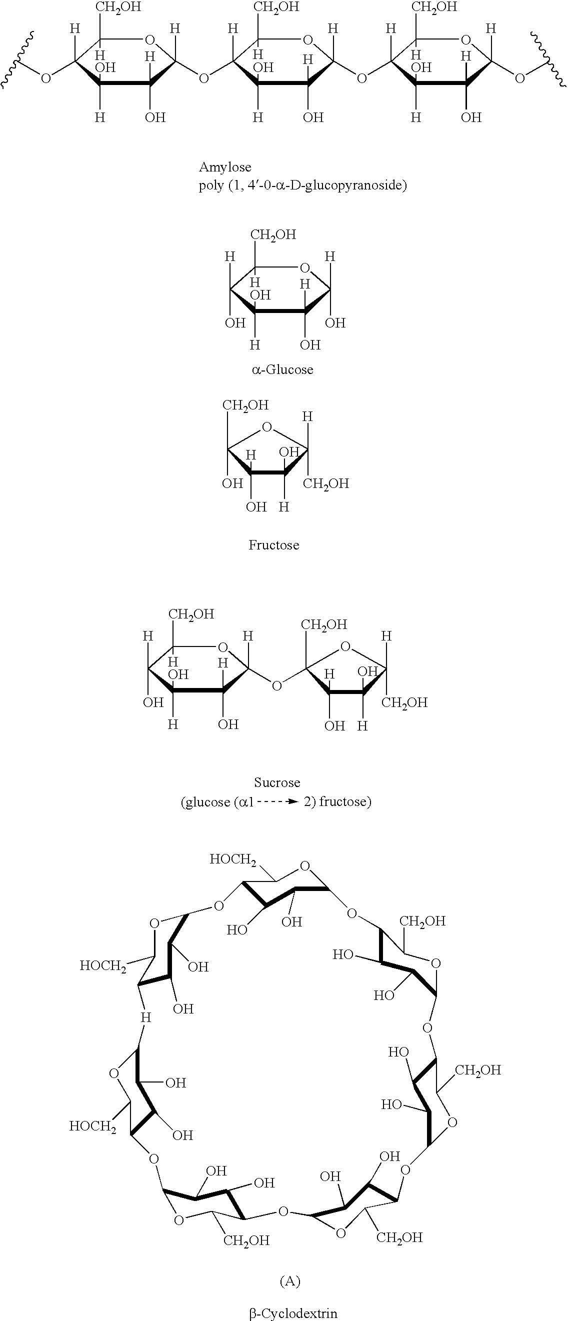

II.C. Carbohydrate-based Materials

In some embodiments of the present invention, a carbohydrate-based material is employed. In the context of the present invention, a carbohydrate-based material comprises a carbohydrate and a non-fluorous CO₂-philic group, and is soluble in one or more forms of carbon dioxide. Preferred carbohydrate-based materials are naturally occurring, although synthetic analogues, as well as other carbohydrate-based materials, can be prepared and are preferably described by the formula:

CₗOₘHₙ₋ᵥRₙ₋ᵥ

wherein:

l ranges from 1 to 100,000;

m ranges from 1 to 100,000;

n ranges from 1 to 100,000;

v ranges from 1 to 100,000;

R is selected from the group consisting of Lewis base groups, such as carbonyl, (as is found in an acetate group or a benzoyl group) and can be generally described as:(C═O)—R₁ wherein R₁ is H, an unsaturated alkyl group, aryl group or a saturated alkyl group, such as —(CH₂)ₚCH₃, wherein p ranges from 0 to 50, sulphonyl and phosphonyl group.

Examples of carbohydrate-based materials suitable for use in accordance with the present invention include without limitation:

Glucose pentaacetate

Galactose pentaacetate

Sorbitol hexaacetate

Sucrose octaacetate

Starch acetate

Cellulose acetate

Cyclodextrin acetate

Glucose pentabenzoate

Sucrose octabenzoate

A carbohydrate-based material (e.g. a CO₂-philic material) can comprise a large polymer, a closed molecule such as a dendrimer, a cluster compound, and a CO₂-philic group. Such materials can be employed for example, as surfactants, ion channels, metal chelates, excepients for drugs, and molecular entrapment materials in carbon dioxide solvent systems, as CO₂ sorbents, or as a CO₂ induced melt. These materials can be employed in a number of applications as disclosed herein.

One example of a carbohydrate-based material of the present invention comprises the general formula:

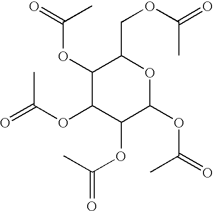

wherein R₁, R₂, R₃, R₄, and R₅ are H atoms or alkyl groups. R₁, R₂, R₃, R₄, and R₅ can each be individually selected and preferably are selected from H, CH₃—, CH₃CH₂—, CH₃ (CH₂)ₙ—, where n=1 to 10. A carbohydrate-based material of the present invention can be present in various amounts relative to carbon dioxide in a system in which carbon dioxide is employed as a solvent. In a preferred embodiment, a carbohydrate-based material comprises from about 0.01, 1, 5, 10, 20, 30, or 40 to about 60, 70, 80, 90, 95, or 99 percent by weight of a system comprising a carbohydrate-based material and a carbon dioxide solvent.

II.D. Lewis Acids and Bases

In one aspect of the present invention, a composition is disclosed and comprises a carbohydrate-based material dispersed in carbon dioxide. The solubility of a carbohydrate, which is normally insoluble in carbon dioxide, is due, in part, to the presence of a CO₂-philic group on the carbohydrate. A CO₂-philic group preferably comprises a Lewis base group. As discussed hereinbelow, the Lewis base group interacts with the carbon atom of carbon dioxide, which assists in associating the carbohydrate with the carbon dioxide.

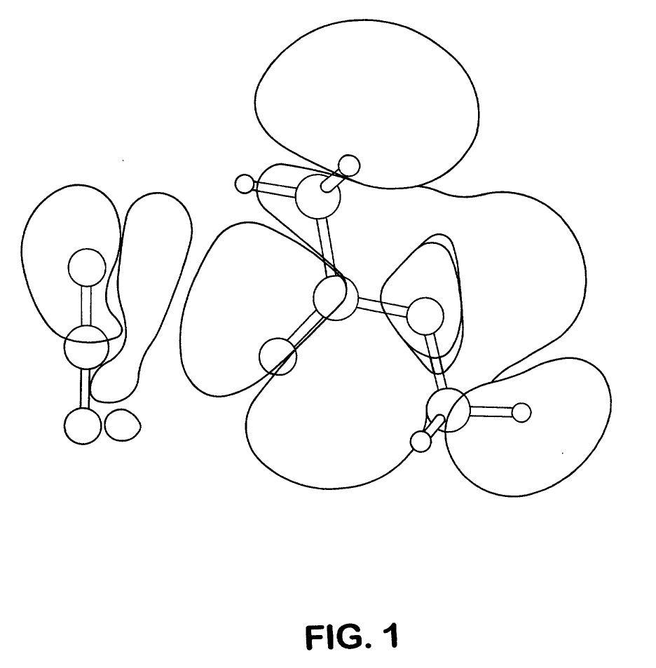

Ab initio calculations have shown that in the case of carbonyl systems having hydrogen atoms attached to a carbonyl carbon or an R-carbon atom, as in an aldehyde or acetate group, a weak, but cooperative C—H O interaction involving these types of hydrogens and one of the oxygen atoms of CO₂ reinforces the LA-LB interactions. The cooperativity of these two interactions is illustrated in FIG. 1.

FIG. 1 is a diagram depicting the highest occupied molecular orbital (HOMO) for the optimized geometry of a CO₂-methyl acetate complex, as calculated by ab initio methods using Gaussian 98 program at the MP2/6-31+G* level. The C—H O hydrogen bond acts cooperatively with the Lewis acid-Lewis base interaction (CO₂-carbonyl) and introduces further stabilization of the carbohydrate-carbon dioxide association.

II.E. CO₂-philic Groups

A carbohydrate-based material of the present invention can comprise a CO₂-philic group comprising a Lewis base. Representative methods of substituting a group on a carbohydrate (e.g. a hydroxyl group or a ring hydrogen) with a group comprising a Lewis base are disclosed. For example, a hydroxyl group of a carbohydrate can be replaced with an acetate group or a benzoyl group by an esterification reaction. A Lewis base group can be removed from a larger compound comprising a Lewis base group. Alternatively, compounds comprising a Lewis base group can be synthesized, and many are available commercially. A representative, but non-limiting list of compounds comprising a Lewis base that can serve as a source for a Lewis base group includes, but is not limited to:

esters such as methyl formate, ethyl formate, butyl formate, isobutyl formate, pentyl formate, methyl acetate, ethyl acetate, propyl acetate, isopropyl acetate, butyl acetate, isobutyl acetate, pentyl acetate, isopentyl acetate, hexyl acetate, cyclohexyl acetate, benzyl acetate, 3-methoxybutyl acetate, 2-ethylbutyl acetate, 3-ethylhexylacetate, 3-methoxybutyl acetate, methyl propionate, ethyl propionate, butyl propionate, isopentyl propionate, methyl butyrate, ethyl butyrate, butyl butyrate, isopentyl butyrate, isobutyl isobutyrate, ethyl isovalerate, isobutyl isovalerate, butyl stearate, pentyl stearate, methyl benzoate, ethyl benzoate, propyl benzoate, butyl benzoate, isopentyl benzoate, benzyl benzoate, ethyl cinnamate, diethyl oxalate, dibutyl oxalate, dipentyl oxalate, diethyl malonate, dimethyl maleate, diethyl maleate, dibutyl maleate, dimethyl phthalate, diethyl phthalate, dibutyl phthalate, diisobutyl phthalate, and triacetin;

amines such as methylamine, dimethylamine, trimethylamine, ethylamine, diethylamine, triethylamine, propylamine, diisopropylamine, butylamine, isobutylamine, dibutylamine, tributylamine, pentylamine, dipentylamine, tripentylamine, 2-ethyihexylamine, allylamine, aniline, N-methylaniline, N,N-dimethylaniline, N,N-diethylaniline, toluidine, cyclohexylamine, dicyclohexylamine, pyrrole, piperidine, pyridine, picoline, 2,4-lutidine, 2,6-lutidine, 2,6-di(t-butyl) pyridine, quinoline, and isoquinoline;

ethers such as diethyl ether, dipropyl ether, diisopropyl ether, dibutyl ether, dihexyl ether, anisole, phenetole, butyl phenyl ether, methoxytoluene, benzyl ethyl ether, diphenyl ether, dibenzyl ether, veratrole, 2-epoxypropane, dioxane, trioxane, furan, 2,5-dimethylfuran, tetrahydrofuran, tetrahydropyrane, 1,2-diethoxyethane, 1,2-dibutoxyethane, and crown ethers;

ketones such as acetone, methyl ethyl ketone, methy propyl ketone, diethyl ketone, butyl methyl ketone, methyl isobutyl ketone, methyl pentyl ketone, dipropyl ketone, diisobutyl ketone, cyclohexanone, methylcyclohexanone, and acetophenone;

thioethers such as dimethyl sulfide, diethyl sulfide, thiophene, and tetrahydrothiophene;

silyl ethers such as tetramethoxysilane, tetraethoxysilane, tetra(n-propoxy)silane, tetra(isopropoxy)silane, tetra(n-butoxy)silane, tetra(isopentoxy)silane, tetra(n-hexoxy)silane, tetraphenoxysilane, tetrakis(2-ethylhexoxy)silane, tetrakis(2-ethylbutoxy)silane, tetrakis(2-methoxyethoxy) silane, methyltrimethoxysilane, ethyltrimethoxysilane, n-propyltrimethoxysilane, isopropyltrimethoxysilane, n-butyltrimethoxysilane, isobutyltrimethoxysilane, sec-butyltrimethoxysilane, t-butyltrimethoxysilane, phenyltrimethoxysilane, vinyltrimethoxysilane, norbornyltrimethoxysilane, cyclohexyltrimethoxysilane, chloromethyltrimethoxysilane, 3-chloropropyltrimethoxysilane, chlorotrimethoxysilane, triethoxysilane, methyltriethoxysilane, ethyltriethoxysilane, n-propyltriethoxysilane, n-butyltriethoxysilane, phenyltriethoxysilane, vinyltriethoxysilane, 3-aminopropyltriethoxysilane, ethyltri(isopropoxy)silane, isopentyl(n-butoxy)silane, methyl(tri-n-hexoxy)silane, methyldimethoxysilane, diemthyldimethoxysilane, n-propylmethyldimethoxysilane, n-propylethyidimethoxysilane, di(n-propyl)dimethoxysilane, isopropylmethyidimethoxysilane, di(isopropyl)dimethoxysilane, n-propylisopropyidimethoxysilane, n-butylmethyldimethoxysilane, n-butylethyidimethoxysilane, n-butyl-n-propyldimethoxysilane, n-butylisopropyldimethoxysilane, di(n-butyl) dimethoxysilane, isobutylmethyidimethoxysilane, diisobutyldimethoxysilane, sec-butylethyidimethoxysilane, di(sec-butyl)dimethoxysilane, t-butylmethyldimethoxysilane, t-butyl-n-propyidimethoxy-silane, di(t-butyl)dimethoxysilane, t-butyl-n-hexyldimethoxysilane, diisoamyldimethoxysilane, n-hexyl-n-propyidimethoxysilane, n-decylmethyidimethoxysilane, norbornylmethyldimethoxysilane, cyclohexylmethyldimethoxysilane, methylphenyidimethoxysilane, diphenyldimethoxysilane, dicyclopentyidimethoxysilne, dimethyidiethoxysilane, diethyldiethoxysilane, di(isopropyl)diethoxysilane, sec-butylmethyidiethoxysilane, t-butylmethyldiethoxysilane, dimethyl(n-butoxy)silane, trimethylmethoxysilane, trimethylethoxysilane, t,methylisopropoxysilane, trimethyl-n-propoxysilane, trimethyl-t-butoxysilane, trimethylisobutoxysilane, trimethyl-n-butoxysilane, trimethyl-n-pentoxysilane, and trimethylphenoxysilane;

phosphines such as methylphosphine, ethylphosphine, phenylphosphine, benzylphosphine, dimethylphosphine, diethylphosphine, diphenylphosphine, methylphenylphosphine, trimethylphosphine, triethylphosphine, triphenylphosphine, tri(n-butyl) phosphine, ethylbenzylphenylphosphine, ethylbenzylbutylphosphine, trimethoxyphosphine, and diethylethoxyphosphine;

phosphine oxides such as triphenylphosphie oxide, dimethylethoxyphosphie oxide, and triethoxyphosphine oxide;

nitriles such as acrylonitrile, cyclohexanedintirile, and benzonitrile;

nitro compounds such as nitrobenzene, nitrotoluene, and dinitrobenzene;

acetals such as acetone dimethylacetal, acetophenone dimethylacetal, benzophenone dimethylacetal, and cyclohexanone dimethylacetal;

carbonate esters such as diethyl carbonate, diphenyl carbonate, and ethylene carbonate;

and thioacetals such as 1-ethoxy-1-(methylthio)cyclopentane, thioketones such as cyclohexanethione.

Difunctional Lewis bases can also be employed, such as, for example, 1,2-di-methoxyethane and N,N,N′,N′-tetramethylethylenediamine, as well as monofunctional Lewis bases, such as, for example, tetrahydrofuran or triethylamine. Monoamines, polyamines, polyhydroxy compounds, reactive polyethers, and polar aprotic compounds, such as ethers and tertiary amines can also be employed as a Lewis base in the compositions and methods of the present invention.

It is noted that the above list of Lewis base group-containing compounds is only representative and additional Lewis base group-containing compounds will be known to those of ordinary skill in the art upon consideration of the present disclosure.

III. A Composition Comprising a Carbohydrate-based Material Dispersed in Carbon Dioxide

In one aspect, the present invention relates to a composition. The composition comprises a carbohydrate-based material dispersed in carbon dioxide, wherein the carbohydrate-based material comprises a carbohydrate and at least one CO₂-philic group. The carbohydrate can be CO₂-philized by the substitution of a functional group of the carbohydrate (e.g. a hydroxyl group or a ring hydrogen) with another functional group, namely a CO₂-philic group. Representative CO₂-philic groups include, for example, acetyl groups and benzoyl groups. Other CO₂-philic groups are listed hereinabove. Any group or moiety comprising a Lewis base group can comprise a CO₂-philic group.

Although substitution can involve any functional group on the carbohydrate and any other functional group, it is preferable that the substitution reaction involves an acetylation reaction or a benzoylation reaction. Preferred reactions lead to at least one hydroxyl group or a ring hydrogen on the carbohydrate-based material being modified, substituted and/or functionalized with at least one CO₂-philic group. Functionalizing a carbohydrate-based material with a CO₂-philic group makes the carbohydrate-based material soluble in the carbon dioxide, absorb carbon dioxide and undergo deliquescence in carbon dioxide or, alternatively, has the ability to absorb/adsorb carbon dioxide without exhibiting deliquescence. Thus, although some forms of carbon dioxide are more preferable for some applications, unless specifically noted, the term carbon dioxide refers to all forms of carbon dioxide, namely supercritical carbon dioxide, liquid carbon dioxide and gaseous carbon dioxide.

IV. Method of Forming a Composition Comprising a Carbohydrate-based Material Dispersed in Carbon Dioxide

In one aspect of the present invention, a method of forming a composition comprising a carbohydrate-based material dispersed in carbon dioxide is disclosed. In a preferred embodiment, the method comprises first providing a CO₂-phobic carbohydrate comprising one or more hydroxyl groups or ring hydrogens. Generally, most unmodified carbohydrates (e.g. those not functionalized with a CO₂-philic group) are CO₂-phobic. Examples of common CO₂-phobic carbohydrates include glucose and galactose. In accordance with the composition described above, a CO₂-phobic carbohydrate can comprise any form of carbohydrate, for example, a CO₂-phobic carbohydrate can be cyclic or acyclic, simple or complex, a monosaccharide or a polysaccharide.

Next, a hydroxyl group or a ring hydrogen is chemically replaced with a CO₂-philic group to form a carbohydrate-based material. A CO₂-philic group preferably comprises a Lewis base group. By substituting a CO₂-philic group for a hydroxyl group or a ring hydrogen, the carbohydrate becomes a carbohydrate-based material able to interact with carbon dioxide (e.g. become soluble in liquid or supercritical CO₂ or exhibit deliquescence with respect to gaseous CO₂ or can absorb or adsorb CO₂).

Lastly, the carbohydrate-based material is dispersed in carbon dioxide. The dispersion can be accomplished by any method. For example, when the carbon dioxide is liquid or supercritical carbon dioxide, the carbohydrate-based material can be dispersed by contacting a carbohydrate-based material with the carbon dioxide, optionally accompanied by agitation. When the carbon dioxide is gaseous, the dispersion can be accomplished by passing gaseous carbon dioxide over the surface of the carbohydrate-based material. Alternatively, the carbohydrate-based material can be introduced into a system comprising gaseous carbon dioxide.

IV.A Preparing a Carbohydrate-based Material

In one aspect of the present invention, a carbohydrate-based material can be prepared. Broadly, a carbohydrate-based material comprises a carbohydrate and a CO₂-philic group. Preferably a CO₂-philic group comprises a Lewis base group.

A carbohydrate-based material can be prepared by substituting a CO₂-philic group for a hydroxyl group or a ring hydrogen present on a carbohydrate. By way of specific example, two different methods of preparing a carbohydrate-based material are discussed hereinbelow, namely acetylation of a carbohydrate and esterification of a carbohydrate. Other substitutions (e.g. benzoylation of a carbohydrate) can be performed by employing chemical methods that will be known to those of ordinary skill in the art upon consideration of the present disclosure.

IV.B. Modification of a Carbohydrate

Ab initio calculations on simple carbonyl systems revealed that methyl acetate has a strong interaction with CO₂ (2.82 kcal/mol at the MP2/aug-cc-pVDZ level). The calculations were carried out using the Gaussian 98 software package. This observation indicates that the acetylation of hydroxyl groups is a viable approach to CO₂-philize hydroxylated compounds (e.g carbohydrates), thereby increasing their solubility. For example, one acetylated carbohydrate that is soluble in CO₂ is sucrose octaacetate. Sucrose octaacetate is very bitter in taste and can be employed as a denaturant for alcohol, a soaker for paper, as well as an insecticide and a plasticizer for cellulosic synthetic resin. It also can be used as an additive for paint and children's toys. When added to these types of items, sucrose octaacetate can deter animals and children from biting or tasting the goods due to its extreme bitter taste.

The abundance of hydroxyl groups in carbohydrates opens a wide range of possibilities for the synthesis of CO₂-philes at reasonable cost. Methods of acetylating a compound, including a carbohydrate, are known in the art and are disclosed herein.

Another modification that can be performed on a carbohydrate to enhance its CO₂-philicity, and thus its solubility, is benzoylation. Benzoylation of a carbohydrate can also enhance its CO₂-philicity. Benzoylation of carbohydrates can also give rise to compounds of commercial interest, which are also soluble in CO₂. An example of a benzoylated carbohydrate that is soluble in CO₂ is sucrose benzoate. Sucrose benzoate is a stable, odorless and glassy solid or white powder. It has excellent ultraviolet light stability. It is compatible with a broad range of resins, plasticizers and solvent. Sucrose benzoate is used in ink industry, as a coating, as a modifier and as a plasticizer for plastics.

Thus, in one aspect of the present invention, a carbohydrate can be subjected to chemical modification. As used herein, the term “chemical modification,” and grammatical derivatives thereof, is used in its broadest sense and encompasses the addition, removal or substitution of a chemical moiety forming an element of a carbohydrate. For example, the term encompasses the addition or removal of a functional group. The term also encompasses the alteration of an element of a carbohydrate, for example, by performing an operation whereby the number or location of chemical bonds is altered.

Such a modification can be known or predicted to alter not only the chemical composition of a carbohydrate, but the chemical and physical properties of the carbohydrate as well. Examples of physical and chemical properties that can be altered by a given chemical modification can include, but are not limited to, a change in the solubility of a carbohydrate with respect to carbon dioxide, a change in the ability of a carbohydrate to adsorb gaseous carbon dioxide, a change in the polarity of a carbohydrate, a change in the hydrophilicity or hydrophobicity of a carbohydrate, the ability of the carbohydrate to form hydrogen bonds, the ability of adsorb a given material and the wettability of the carbohydrate.

A chemical modification of a carbohydrate can be any chemical modification. In a preferred example, a carbohydrate can be esterified. In another preferred example, a carbohydrate can be acetylated.

There is no limit on the number of chemical modifications that can be performed. For example, a carbohydrate that has been acetylated can itself be the subject of subsequent chemical modification and can be modified to include, for example, alkyl chains and polar functional groups.

IV.B.1 Acetylation of a Carbohydrate

In one embodiment, a carbohydrate-based material can be prepared according to the following synthetic scheme. In this example, a carbohydrate is acetylated. Generally, a carbohydrate can be acetylated by refluxing it with an equimolar mixture acetic acid and acetic anhydride for several hours or in a biphasic CO₂ based solvent system.

For example:

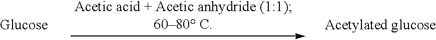

Other methods of acetylating a carbohydrate, as well as variations on this method, can also be employed for making a carbohydrate-based material and will be known to those or ordinary skill in the art, upon contemplation of the present disclosure.

IV.B.2. Esterification of a Carbohydrate

In another embodiment, a carbohydrate-based material can comprise two or more carbohydrate units esterified to form a single unit. In this method of preparing a carbohydrate-based material, two or more carbohydrate units can first be functionalized via acetylation. Acetylation, as described hereinabove, generally involves substitution of one or more hydroxyl groups or ring hydrogens of a carbohydrate with an acetyl group. This step makes the carbohydrate CO₂-philic and any subsequent steps can be performed using carbon dioxide as a solvent.

After acetylation of a carbohydrate, a polymerizable group, such as, for example, allyl or vinyl groups can be introduced into an acetylated carbohydrate. This form of carbohydrate-based material has high solubility in liquid and scCO₂. Polymerization can be initiated via a free radical initiator such as, for example, 2,2′-azobisisobutyronitrile (AIBN) or by an enzyme. Formed carbohydrate-based material polymers typically have lower solubility in CO₂ and can separate out of solution spontaneously upon formation. Thus, in this embodiment of the present invention it is possible to separate polymers of different lengths, which can be achieved by adjusting the CO₂ pressure.

When performing an esterification polymerization reaction according to the present invention, an allyl substitution (i.e. replacing a hydrogen or hydroxyl group with a carbohydrate monomer) can be at any of the sugar ring carbons. It is preferable, however, that the substitution is directed to either the C-2 and/or the C-6 positions. The following reaction scheme demonstrates one method of forming a polymeric carbohydrate species, which employs carbon dioxide as a solvent.

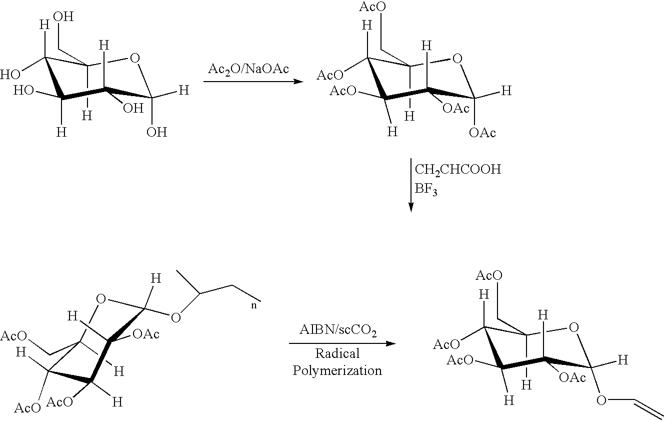

IV.B.3. Benzoylation

Another preferred method of functionalizing (e.g. CO₂-philizing) a carbohydrate is by introducing a Lewis base group into the carbohydrate via benzoylation of the carbohydrate. For example, glucose can be benzoylated using benzoyl chloride in the presence of triethylamine:

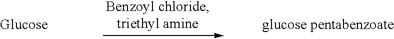

Additional methods of benzoylating a carbohydrate will be known to those of ordinarily skill in the art, upon consideration of the present disclosure.

V. Applications

The compositions of the present invention are extremely versatile and can be used in a wide variety of applications. Such applications include, but are not limited to, densifying carbon dioxide by the addition of a composition of the present invention (i.e. modulating the viscosity of carbon dioxide by employing a composition of the present invention), sequestering carbon dioxide from a CO₂ source, such as for example, effluent from a fossil fuel burning system, natural product extraction, preparation of a CO₂-philic surfactant for making reverse and normal microemulsions, as well as other surfactant uses in CO₂, extraction of proteins and gene transfection agents, metal ion extractions (i.e. metal chelation), homogeneous and heterogeneous polymerizations, homogeneous and heterogeneous catalysis, and membrane and separation support media synthesis.

A composition of the present invention can also be employed in the preparation of nanomaterials (including nanoparticles and assemblies of nanoparticles) that are soluble or insoluble in CO₂. Nanomaterial synthesis methods in which the present invention can be of particular use include those involving GAS (gas anti-solvent) and RESS (Rapid Expansion of Supercritical Solutions) methods. Other applications of the present invention include micronization applications, as well as in applications in the food, cosmetic, pharmaceutical, and biopolymer industries.

The compositions can also be used in sizing and desizing textiles and paper products in liquid and supercritical CO₂, in which both the solvent and the size can be completely recycled. Additionally, known sizes suitable for use in a CO₂-based system (see, e.g., U.S. Pat. No. 5,863,298) are expensive and economically impractical. The carbohydrate materials disclosed in the present invention can serve as inexpensive, renewable size materials. In these application, both the solvent and the size material are environmentally benign and thus eliminates the environmental hazards.

Due to the high solubility of these materials in CO₂ and their high affinity for CO₂, CO₂ can be used for separating, purifying, and crystallizing sugar esters and their derivatives, and in the synthesis and separation of carbohydrate-based biodegradable polymers based on these materials.

Some of these materials, such as for example glucose pentaacetate, undergoes photolysis in CO₂ absorbing UV radiation, and these materials thereafter can be used as free radical initiators in CO₂ for polymerization processes, bleaching compositions and other photochemical processes.

Some of the materials described above undergo deliquescence in CO₂ and the CO₂ melt of these materials can be used to make shaped glassy materials for various applications. Additionally, these melts can be employed as a dispersion medium for dispersing molecules or ions or atoms therein.

Carbon dioxide can be used for dispersing other molecules, such as drugs, and compounds comprising carbohydrate esters (as an excepient or a carrier).

Also, photographic materials such as silver halides can be dispersed in carbohydrate derivatives such as sucrose octaacetate using liquid and scCO₂ as the dispersing solvent to prevent crystallization of the reduced silver.

Some of these materials, such as acetylated carbohydrates and benzoylated carbohydrates, have a number of applications, and can form a component of insect repellants, bitter taste additives, bitter coatings, plasticizer for cellulosic and non-cellulosic materials, soaker for paper, rat repellants etc. By virtue of their high solubility in CO₂, CO₂ can be employed as a medium for dispersing or impregnating these materials for example in wood, paper, and yarn. Carbon dioxide can also be employed to disperse these materials, which can subsequently be sprayed out to produce thin films or nano-sized or micron-sized particles.

Several of these applications of the present invention are described more fully hereinbelow. Those of ordinary skill in the art will recognize that a discussion presented in the context of one application can be employed mutatis mutandis in other applications. Thus, the following discussion of several applications can be employed in other applications as well.

V.A. Viscosity Modulation

The present discovery is related to the identification of a new class of inexpensive, non-hazardous, agriculturally based, renewable materials having extreme solubility in liquid and supercritical carbon dioxide that can be employed as densifiers for carbon dioxide in a number of industrial processes. Densification and viscosity enhancement of liquid and supercritical carbon dioxide has gained considerable attention the recent past due to its application in the oil and gas industry. There are at least two processes in these industries that employ densified carbon dioxide: enhanced oil recovery (EOR) and fracture stimulation. Both these processes are designed to increase the production of oil from a reservoir.

In these processes, carbon dioxide acts as a medium that can be employed to separate crude oil from the porous rock in which it resides. In practice, carbon dioxide can be injected into an oil reservoir to recover oil left behind during water flooding. This enhanced oil recovery technique is commonly referred to as “miscible displacement.”

During a miscible displacement project, carbon dioxide dynamically develops miscibility as it mixes with the oil in the porous media. This process is conducted at or just above a “minimum miscibility pressure,” to ensure high degree of solvency for the oil it contacts. As the reservoir fluids are produced from the reservoir, the carbon dioxide can be readily separated from the oil and brine by pressure reduction.

In an EOR process, carbon dioxide enters the oil bearing porous media at the reservoir temperature, generally at about 80-250° F. A disadvantage of CO₂ as oil displacement fluid is its low viscosity (about 0.03-0.1 cp) compared to the fluid it is displacing. The CO₂ slug therefore has a much higher mobility than the fluid it is displacing. As a result, the real sweep efficiency is reduced as CO₂ fingers towards the production wells, rather than uniformly displacing the oil ahead of it toward the production wells. Consequently, if the viscosity of the carbon dioxide can be increased to a level comparable with the oil it is displacing, typically a 1-2 order of magnitude increase, substantial improvements in the sweep efficiency and oil recovery can be achieved.

Another petroleum engineering technology that employs dense carbon dioxide is the fracturing of gas and oil wells. Carbon dioxide-rich mixtures have been used for fracture clean-up and sand fracturing of wells. It has been suggested that densification of carbon dioxide can increase its fracturing efficiency.

However, a limitation of this approach is the lack of inexpensive materials having high solubility in CO₂ that can increase the viscosity of carbon dioxide. The currently available CO₂-philes are the expensive fluorocarbons and siloxanes, which are not only cost effective, but also are not soluble enough to densify carbon dioxide to the required proportions.

In one aspect of the present invention, a class of carbohydrate-based materials having extreme solubility in liquid and supercritical carbon dioxide is disclosed. This class of compounds can be employed, for example, to tune the density and viscosity of carbon dioxide to any desired level. Also, these carbohydrate-based materials can be easily functionalized with long alkane chains or self-associating functional groups to increase miscibility with oil. Such functionalizations can make operating conditions simpler by reducing the miscibility pressures and increasing the processing efficiency.

Thus, in one aspect, the present invention discloses a new class of inexpensive, non-hazardous, agriculturally based, renewable carbohydrate-based materials having extreme solubility in liquid and supercritical carbon dioxide that can be used as densifiers for carbon dioxide.

In one aspect, a composition of the present invention can be employed to modulate the viscosity of carbon dioxide. In this application, a carbohydrate-based material adapted for dispersion in carbon dioxide is provided. Preferably, a carbohydrate-based material comprises one or more CO₂-philic groups, which has been substituted for a hydroxyl group or a ring hydrogen. Preferably a CO₂-philic group(s) comprises a Lewis base group.

Suitable carbohydrate-based materials can be synthesized by employing the methods disclosed herein. For example, as described herein, a carbohydrate-based material can be prepared by acetylating or benzoylating a carbohydrate, which has the effect of making the carbohydrate soluble (or more soluble) in CO₂. Representative carbohydrate-based materials include, but are not limited to AGLU, BGLU and BGLA. Indeed, any carbohydrate-based material comprising a carbohydrate and a CO₂-philic group can be employed in a method of modulating viscosity.

Next, an amount of the carbohydrate-based material is dispersed in a composition comprising carbon dioxide sufficient to modulate the viscosity of the composition comprising carbon dioxide to a desired viscosity. The dispersion can be performed by dissolving the required amount of the carbohydrate-based material in CO₂.

V.B. Preparation of a Surfactant

A carbohydrate or carbohydrate-based material can be modified to function as a surfactant by attaching a polar functional group to a carbohydrate (e.g. linked through an alkyl chain) as in —(CH₂)_{q}Y wherein q ranges from 0 to 50; and Y is a polar functional group such as, for example, —COOH, —SH, —OH, —N(CH₃)₃⁺, SO₃⁻, —PO₃⁻, or their derivatives in the neutral or ionic form; and metal salts and coordination complexes of compounds comprising these groups. The polar functionality can also be linked to a carbohydrate as in —X(CH₂)Y, wherein X is a heteroatom such as, for example, N, S or P.

If a CO₂-philic functionality attached to a carbohydrate is an acetate group, then some surfactants that can be prepared can be generally described as: p1 G—X—(CH₂)_{q}COOH (where X is, for example, NH, O, S, P, etc.)

G—X—(CH₂)_{q}CH₃

G—X—(CH₂)_{q}—N(R)₃⁺

wherein q ranges from 0 to 50; and G is a Lewis-base functionalized CO₂-philic carbohydrate such as, for example, acetylated glucose, acetylated sucrose, acetylated cyclodextrin, and sucrose benzoate. The CO₂-phobic group of a surfactant of the present invention can comprise any head group, including, but not limited to, hydrogen, a carboxylic acid group, a hydroxy group, a phosphato group, a phosphato ester group, a sulfonyl group, a sulfonate group, a sulfate group, a branched or straight chained polyalkylene oxide group, an amine oxide group, an alkenyl group, a nitryl group, a glyceryl group, an aryl group unsubstituted or substituted with an alkyl group or an alkenyl group, a carbohydrate unsubstituted or substituted with an alkyl group or an alkenyl group, an alkyl ammonium group, or an ammonium group. A carbohydrate can comprise, for example, sugars, such as sorbitol, sucrose, or glucose. A CO₂-phobic region of a surfactant can comprise an ion, such as, for example, H⁺, Na⁺, Li⁺, K\NH\Ca, Mg²⁺, Cl⁻, Br⁻, I⁻, mesylate and tosylate. A CO₂-phobic region of the surfactant can also comprise a non-acetylated (or hydroxylated) sugar.

Synthesis of a surfactant for CO₂/water or CO₂/organic interfaces is a challenging area in supercritical fluid research. A surfactant preferably comprises a CO₂-philic region, as well as a CO₂-phobic region. A carbohydrate-based material, as disclosed herein, can comprise a CO₂-philic region of a surfactant.

In one embodiment, an acetylated carbohydrate can be employed as a CO₂-philic group in a surfactant. Such surfactants can be prepared by chemically associating a CO₂-phobic region to the aCO₂-philic group. These surfactants can be employed in the formation of water-in-CO₂ microemulsions in CO₂ and can solubilize polar materials in the water core of formed reverse micelles. This method can be employed in analytical extractions, such as the extraction of polar biomolecules, (e.g. proteins), using carbon dioxide as the principal medium.

In a surfactant, a polar head group is preferably attached to a CO₂-philic carbohydrate-based material via an alkyl chain. A surfactant can comprise one or more CO₂-philic units. A surfactant can be a single chain or double chain type surfactant. A CO₂-phobic region of a surfactant of the present invention can comprise any head group commonly found in a surfactant, including, but not limited to, hydrogen, a carboxylic acid group, a hydroxy group, a phosphato group, a phosphato ester group, a sulfonyl group, a sulfonate group, a sulfate group, a branched or straight chained polyalkylene oxide group, an amine oxide group, an alkenyl group, a nitryl group, a glyceryl group, an aryl group unsubstituted or substituted with an alkyl group or an alkenyl group, an alkyl ammonium group, or an ammonium group. A CO₂-phobic part of a surfactant can also comprise a non-acetylated (or hydroxylated) carbohydrate. Preferred carbohydrates groups can include, for example, sugars such as sorbitol, sucrose, or glucose. A CO₂-phobic group can likewise include an ion selected from the group of H⁺, Na⁺, Li⁺, K\NH\Ca, Mg²⁺, Cl⁻, Br, I⁻, mesylate and tosylate. The CO₂-phobic region can also comprise an alkyl chain, which will form a surfactant for organic-in CO₂ reverse microemulsions.

V.C. Metal Chelation

Due, in part, to their favorable properties, which includes variable solvent power and low viscosity, supercritical fluids have been employed in a variety of selective extraction processes. Although a number of common gases exhibit desirably low critical temperatures (below 100° C.), carbon dioxide is one of the most widely used solvents in supercritical fluid science and technology. See, e.g., McHugh & Krukonis, (1986) Supercritical Fluid Extraction, Butterworths, Stoneham, Mass., United States of America. Carbon dioxide is readily available, inexpensive, relatively non-toxic, non-flammable, and exhibits a critical temperature of about 31° C., which is lower than many other gases. Carbon dioxide is also one of the few organic solvents that occur naturally in large quantities. Moreover, because CO₂ is a gas under ambient conditions, reduction of liquid or supercritical CO₂-based solutions to atmospheric pressure induces essentially complete precipitation of solute, thereby facilitating solute/solvent separation.

At present, the poor solubility of conventional chelating agents in CO₂ has prevented process extraction of metals using such chelating agents in CO₂. Due to the advantageous properties of CO₂ described above, however, it is desirable to develop chelating agents, and methods for making the chelating agents, for performing such extractions. In one aspect, the present invention solves this problem by disclosing methods and compositions adapted to chelate metals.

Although chelation of metals is known, (see, e.g., U.S. Pat. No. 6,187,911), the high cost of the CO₂ soluble metal chelates and other problems limit the application of this method on an industrial scale. In one aspect, the present invention discloses the synthesis of inexpensive CO₂-soluble metal chelates from carbohydrate materials. As disclosed herein, these methods and compositions comprise employing a carbohydrate, which can be derivatized with a functional group, dispersed in carbon dioxide.

Thus, in accordance with the present invention, a method of chelating a metal atom disposed in carbon dioxide is disclosed. Although it is preferable that a metal atom be free in solution, the methods of the present invention can also be employed when the metal atom is associated with a compound. In a preferred embodiment, the method comprises providing a CO₂-philic carbohydrate-based material comprising a carbohydrate, at least one CO₂-philic group and at least one chelating group covalently linked to one of the CO₂-philic group and the carbohydrate. A carbohydrate-based material can be prepared as described herein.

A chelating group can be added to a carbohydrate-based material by synthetic approaches known to those or ordinary skill in the art upon consideration of the present invention. For example, when a chelating group is added to a ring of a carbohydrate-based material, known carbohydrate chemistry methods can be employed. When a chelating group is added to a CO₂-philic group, consideration of the nature of the CO₂-philic group can assist in designing a strategy for associating the chelating group with the CO₂-philic group.

Next, a carbohydrate-based material, which has been functionalized with a chelating group can be contacted with a sample comprising carbon dioxide, in which a metal atom is known or suspected to be disposed. Preferably conditions conducive to metal chelation (e.g. pH, ion concentration, temperature, etc.) are maintained with respect to the sample.

The contacting can be accomplished by any convenient method, and can depend, in part on the nature and disposition of the sample. For example, if the chelation is performed under controlled conditions, the carbohydrate-based material can be dispersed in the sample, preferably with agitation. In other situations, for example when the sample is an environmental sample and the chelation is performed in the field, the contacting can be carried out in view of the disposition of the sample.

The disclosed method can be used to solubilize a number of functional compounds including but not limited to catalysts and dyes, when it is desirable to solubilize these materials in CO₂.

V.D. Sizing a Substrate

In the textile industry, many current production methods for producing woven fabrics such as high-speed air jet looms require sizing of the yarn. Sizing of yarn occurs when yarn is coated with a material (i.e. a sizing material) in order to improve its strength to withstand high stress and retain high quality. Presently, yarn sizing is done by drawing the yarn through an aqueous solution or colloidal dispersion of a sizing material and then drying the yarn. This method consumes a great deal of energy required for drying the yarn later. The generation of a large amount of wastewater raises environmental issues. The same problems are applicable to the desizing of the yarn also, where the size material is removed by water treatment.

Liquid and supercritical CO₂ (scCO₂) is a viable solvent alternative for sizing and desizing, since very little energy is required for the drying process, which can lead to a reduction in waste (see, e.g., U.S. Pat. No. 5,863,298). Also, an almost complete recyclability of the size material and the solvent are an added advantage favoring the use of liquid and scCO₂-based processes.

However, the application of this carbon dioxide-based method in the textile industry has not been achieved. This is due, in part, to the lack of size materials having high solubility in liquid and scCO₂, as well as the need for high pressure tanks for the sizing operation.

However, the methods and compositions of the present invention are not limited to sizing yarns and other textile-related materials. Indeed, the compositions and methods of the present invention (e.g. acetylated or benzoylated carbohydrates) can be employed to size many different types of materials. For example, paper can be sized. A size can be selected, prepared and delivered using the methods of the present invention. Some sizes, such as sucrose sucrose octaacetate, are presently employed as hydrophobic soakers for paper and other cellulosic and non-cellulosic materials, as well as nsecticides and pest repellants. Depending on the nature of the selected size, the integrity of the paper can be preserved for many years. Sizes can be selected so as to deter damage to the paper by pests or to maintain the integrity and/or intensity of the ink used in printing on the paper and/or the color of matter printed on the paper. Also, materials such as sucrose octaacetate are used as plasticizers and protective materials for wood. By virtue of their high solubility in CO₂, it is possible to employ CO₂ as a solvent or as a medium for dispersing these materials, thereby targeting applications involving impregnation of a material into a substrate material.

Thus, in one aspect, the present invention relates to a class of carbohydrate derivatives (e.g. carbohydrate-based materials) having extreme solubility in CO₂ at low pressures. These materials can be employed as size materials, enabling this low-cost, environmentally benign technology in the textile industry.

In a preferred embodiment of a method of sizing a substrate, the method comprises providing a carbohydrate-based material comprising a carbohydrate, at least one CO₂-philic group and at least one moiety known or suspected to be an effective size. Carbohydrate-based materials can be prepared as described herein. Representative carbohydrates and CO₂-philic groups are also disclosed herein.

The nature of a moiety known or suspected to be an effective size can depend, in part, on the nature of the material that will be sized. For example, when yarn or another textile is sized, preferred moieties known or suspected to be an effective size can comprise, but are not limited to, acetylated carbohydrates. When the material to be sized comprises paper or a paper product, a different form of size can be employed. Thus, when paper is sized, preferred size moieties can comprise acetylated or benzoylated carbohydrates.

After a carbohydrate-based material is provided, the carbohydrate-based material can be dispersed in carbon dioxide to form a sizing solution. Preferably, but not necessarily, the dispersion is accompanied by agitation. Upon dispersal (or melting by CO₂) of the carbohydrate material, a sizing solution is formed as is ready to be employed in a sizing operation.

Next, a substrate is contacted with the sizing solution. The nature of the contacting can be dependent on the nature of the material being sized. For example, when yarn or another textile material is sized, the contacting can be achieved by passing the yarn through a bath comprising the sizing solution one or more times and subsequently spooling the yarn. When paper is being sized, a sizing solution can be sprayed directly onto the paper itself. Alternatively, the paper can be contacted with a size bath. In another embodiment, a size can form a component of a substrate (e.g. yarn or paper) and can be incorporated during the manufacture of the substrate. Other applications in which it might be desirable to introduce a size into a substrate will be apparent to those of ordinary skill in the art upon consideration of the present disclosure.

V.E. Pharmaceutical Applications

The compositions of the present invention can be employed in a range of pharmaceutical applications. For example, the compositions of the present invention can be employed in the formation of water/CO₂ and organic/CO₂ reverse microemulsions. Such microemulsions can be employed in the separation of pharmaceutically relevant and bio-active materials, using liquid and supercritical CO₂ as a solvent (see, e.g., U.S. Pat. No. 5,733,964). Applications in which carbon dioxide is employed as a co-solvent for pharmaceutically important molecules including proteins for example in liquid and supercritical CO₂ are also made possible by the present invention.

In another example, a composition of the present invention (e.g. a carbohydrate-based material) can be employed as a solid diluent (e.g. an excipient) in pharmaceutical formulations. In this application, an active agent (e.g. a pharmaceutical) can be associated with a carbohydrate-based material of the present invention in a desired proportion, and can form an element of a pharmaceutical formulation. Due to the solubility of carbohydrate-based materials in carbon dioxide, an aspect of the present invention, an association or dilution can be carried out that employs CO₂ as a solvent for both the active agent and an excipient. Many pharmaceuticals comprise carbohydrate esters, which are soluble in carbon dioxide or melt on contact with gaseous carbon dioxide, an observation that forms another aspect of the present invention. After an association has been carried out, the carbon dioxide can be easily removed from the system by altering, for example, the pressure and/or temperature conditions of the carbon dioxide.

In yet another example, a compound can be employed to encapsulate an active agent. Encapsulation of materials, particularly active agents and enzymes, in sugar esters (e.g. acetylated cyclodextrins and sucrose octaacetate) can form a basis for temporarily protecting an active agent from degradation in the digestive system of a patient and the protracted time release of an active agent. The encapsulation process can be carried out in a carbon dioxide solvent, which is more benign than the organic solvents conventionally employed for such operations.

In this application of the present invention, an active agent can be dispersed in a CO₂-philic diluent using carbon dioxide as a medium under conditions in which the CO₂-philic diluent or encapsulating agent are soluble in CO₂ or are melted by CO₂. A carbohydrate-based encapsulation material, such as cyclodextrin acetate, can also be dispersed in the carbon dioxide medium. Conditions can be adjusted such that the active agent will preferentially associate with the carbohydrate-based material. After the association has been performed, the CO₂ medium can be removed (e.g. by varying the temperature and pressure conditions associated with the medium).

Upon administration to a patient, the resulting encapsulated active agent can be released in a time-dependent fashion. As the carbohydrate-based encapsulation material is broken down by in the body of a patient, the active agent is gradually released. By selecting an encapsulation material having certain properties, a desired release pattern can be achieved.

In yet another pharmaceutically-related application that forms an aspect of the present invention, nanoparticles comprising an active agent and a carbohydrate-based material employed as an excipient can be prepared. Such nanoparticles can be of particular use in delivering an active agent to a patient and can themselves be useful as a component of a formulation. Nanoparticles comprising a carbohydrate-based material and an active agent can be prepared by co-dispersing the material and the agent in carbon dioxide to form a system. Under certain conditions, the material and the agent will associated, for example, as described above with respect to the encapsulation of an active agent. The carbon dioxide can be rapidly expanded by a rapid change in the temperature or pressure of the system. Under some conditions, this change in the system can volatize the carbon dioxide solvent, leaving only nanoparticles comprising an active agent and the carbohydrate-based material. Similarly, thin films comprising these compounds can be formed the by expansion of the system onto a surface.

V.F. Synthesis Medium

In one aspect, the present invention relates to a carbohydrate-based material that is adapted to be dispersed in carbon dioxide. One particular application of a compound of the present invention is in the synthesis of carbohydrate-based polymers (e.g. biopolymers), which can be performed in liquid and supercritical carbon dioxide. In this application, carbon dioxide can act as a solvent in which a polymerization reaction can be performed.

In one aspect, the present invention discloses a method of synthesizing a polymer in CO₂. Such a polymer can have a wide range of industrial applications, ranging from the food and pharmaceutical industries to the packaging industry and the biomedical industry.

In a preferred embodiment, a method of synthesizing a polymer comprises providing a carbohydrate-based material comprising a CO₂-philic group. A carbohydrate unit is preferably a single carbohydrate molecule, such as glucose. A carbohydrate-based material can, however, comprise a disaccharide or a polysaccharide, such as, for example, sucrose, which comprises a glucose monomer and a fructose monomer joined by a linkage between the anomeric carbons of these monomers. Preferred CO₂-philic groups are disclosed herein and preferably comprise a Lewis base group.

Next, a seed unit can be formed by joining the carbohydrate-based material with a compound comprising a polymerizable group. Preferably the joining is via an ester linkage formed between the carbohydrate and the polymerizable group. Esterification can be achieved by employing synthetic methods known to those of ordinary skill in the art and disclosed herein. Any group adapted for polymerization can be employed, however preferred polymerizable groups comprise organic chemical entities comprising allyl groups, vinyl groups, styrenes, ethylenes and combinations thereof.

A seed unit can then be dispersed in carbon dioxide. The seed unit can be dispersed, for example, by contacting the seed unit with the carbon dioxide with or without agitation. The enhanced solubility of the carbohydrate-based material, in part, makes this dispersion possible.

When the seed unit is dispersed in carbon dioxide, polymerization can be initiated. Polymerization can be initiated by the addition of a free radical initiator, such as AIBN or an enzyme. Polymerization can be allowed to continue under a predetermined set of conditions that offer a measure of control over the degree of polymerization.

Polymers formed by the methods of the present invention will have lower solubility in CO₂ and will separate out spontaneously. Thus, as formed polymers reach a certain length, the polymers will precipitate out of solution and can be recovered by any of a variety of techniques. Another advantage of the present invention is that it is possible to separate out polymers of different polymer lengths based by adjusting the CO₂ pressure. Therefore, adjustment of CO₂ pressure can facilitate the formation of polymers of a desired length. This ability offers a degree of control over the polymerization process not observed in some other polymerization schemes.

Further, cross polymerization of these compounds with other polymerizable monomers offer tremendous possibilities. A representative polymerization scheme is presented below. The following scheme is meant to illustrate a preferred, but not the only embodiment of a polymerization method of the present invention.

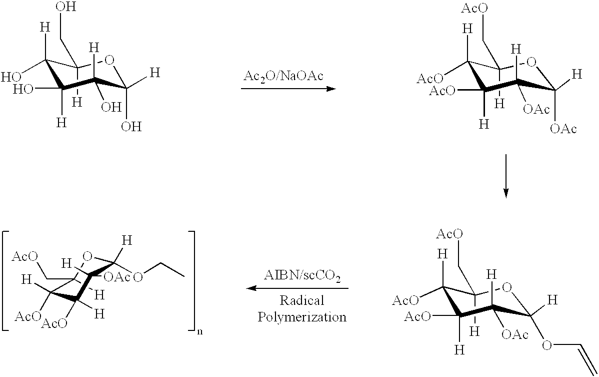

V.G. Sorption of Carbon Dioxide from a Sample

Removal of carbon dioxide from flu gases and other gas streams has been a challenging problem due to its extensive applications in a number of areas including power plants and gas purification systems. The problem of the removal of CO₂ from a sample, which can comprise flu gases, is solved in whole or in part by the methods and compositions of the present invention.

In a preferred embodiment of a method of adsorbing carbon dioxide from a sample, the method comprises first providing a CO₂-philic carbohydrate-based material comprising a carbohydrate and at least one CO₂-philic group. Carbohydrate-based materials comprising a carbohydrate and at least one CO₂-philic group are disclosed herein, as well as methods of preparing such compounds. The compositions of the present invention and thus, those of the present method, preferably comprise CO₂-philic groups that comprise a Lewis base moiety, which, by its nature, is adapted to interact with a Lewis acid moiety.

Continuing with the method, the CO₂-philic carbohydrate-based material is contacted with a sample known or suspected to comprise carbon dioxide. The sample can be known or suspected to comprise liquid, supercritical or gaseous carbon dioxide, although it is preferable that the carbon dioxide takes the form of gaseous carbon dioxide when the sample comprises flu gases. When the sample is gaseous, the sample can be passed over the carbohydrate-based material, which can be arranged in a bed or a column through which the sample passes. For example, a carbohydrate-based material can be disposed in a structure that can be fitted on a flu, such as those found associated with a power plant. A sample, such as combustion gases from an engine or power plant, can then be contacted with the structure. Carbon dioxide in the sample will adsorb to the carbohydrate-based material and be effectively trapped out of the sample, the remainder of which will not interact with the carbohydrate-based material and can exit the system.

The present method can be employed in a range of industrial applications. Indeed, the method can be employed in any application in which it is desired to remove carbon dioxide from a sample or, for example, a sample stream. Further, since a CO₂-philic carbohydrate-based material can interactively stabilize a complex formed between a carbohydrate-based material and gases other than CO₂, such as SO₂ and H₂S. Such complexes can form due to the presence of Lewis base groups in these compounds and samples. Thus, the compositions of the present invention can also be employed in the removal of gases such as SO₂ and H₂S, gases commonly considered pollutants and typically emitted from power plants and factories. In another embodiment, a CO₂-philic carbohydrate-based material can be immobilized on a membrane for efficient separation of CO₂.

V.H. Isolation of a Carbohydrate Ester

Sugar esters have extensive applications in food, pharmaceutical and cosmetic industry since they are non-toxic, edible and easily degradable into naturally occurring materials. Current methods of separation, purification and crystallization of these materials involve the use of organic solvents. In one aspect of the present invention, the present invention discloses a method of employing supercritical, liquid and gaseous carbon dioxide for the extraction of these materials. Carbon dioxide is an environmentally benign, non-toxic and nonflammable solvent, which can be easily removed from the separated products, making it a desirable replacement for the organic solvents typically employed in such extraction operations.

Thus, in one aspect of the present invention, a method of isolating a carbohydrate ester from a sample is disclosed. In a preferred embodiment, the method comprises providing a sample known or suspected to comprise a carbohydrate ester. A representative, but non-limiting, list of samples that can be known or suspected to comprise a carbohydrate ester includes glucose pentaacetate, sucrose octaacetate and galactose pentaacetate. Many of these samples are of commercial relevance.

Continuing with the method, the sample is contacted with carbon dioxide to form an extraction mixture. The method of contacting can take any form and can depend, in part, on the nature of the sample. Upon contacting the sample with the carbon dioxide, any carbohydrate esters present in the sample will become soluble in the carbon dioxide and will partition with the carbon dioxide. This is due, in part, to the discovery that carbohydrate esters are soluble in carbon dioxide, which forms an aspect of the present invention.

Next, the extraction mixture is isolated from the sample. The nature of the isolation operation can again depend, in part, on the nature of the sample. For example, if a sample is a gas, the gas can be passed through or over the carbon dioxide, in which case any carbohydrate esters present therein will remain with the carbon dioxide fraction (i.e. the extraction mixture). In another example, when a sample is volatile, an extraction mixture can be isolated by varying the pressure on or above the carbon dioxide.

In another aspect of the present invention, a method of separating carbohydrate-containing molecules from naturally occurring matrices is disclosed. In a preferred embodiment, carbohydrate-containing molecules to be extracted are CO₂-philized by subjecting the carbohydrate-containing molecules to a CO₂-philization process, such as acetylation or benzoylation. Acetylation can be achieved by treating the carbohydrate-containing molecules with acetic anhydride and acetic acid. This process replaces one or more hydroxyl groups of the carbohydrate with one or more acetyl groups, making the material a CO₂-philic carbohydrate-based material. Next, the matrix containing the acetylated carbohydrate-based material is contacted with CO₂, whereby CO₂-philic carbohydrate-containing molecules are transported into the CO₂ medium. The CO₂-solution can then be depressurized to recover the carbohydrate-containing material. The acetylated carbohydrate-based material can then be hydrolyzed to isolate the molecules of interest.

Additionally, room temperature melting of a carbohydrate-based material can be employed in a number of applications, including, for example, glassification and production of mesoporous materials.

VI. Conclusions

In one aspect of the present invention, a composition is disclosed. The composition comprises a carbohydrate-based material comprising a carbohydrate derivatized with at least one non-fluorous CO₂-philic group. This composition exhibits solubility in carbon dioxide. In another aspect, the present invention discloses the deliquescence of a peracetylated sugar in contact with gaseous CO₂. To the inventors' knowledge, although solubility in carbon dioxide has been observed for some compounds, such solubility has not been observed for a carbohydrate-based material, prior to the present disclosure.

The present invention offers the potential for renewable, biologically derived, nonvolatile materials with high miscibility and solubility in CO₂. The compositions of the present invention can serve as an intermediate in a wide range of carbohydrate chemistries and discloses methods by which liquid and supercritical CO₂ can serve as a unique solvent for reactions as well as analytical and preparative separations in carbohydrate chemistry. Thus, the methods and compositions of the present invention can be employed in many applications, some of which are discussed above. Additional applications based on and/or incorporating the methods and compositions of the present invention will be apparent to those of ordinary skill in the art upon consideration of the present disclosure.

### LABORATORY EXAMPLES

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the present invention.

### Laboratory Example 1

### Solubility Behavior of β 1,2,3,4, 6-Pentaacetyl β-D-Glucose (BGLU)

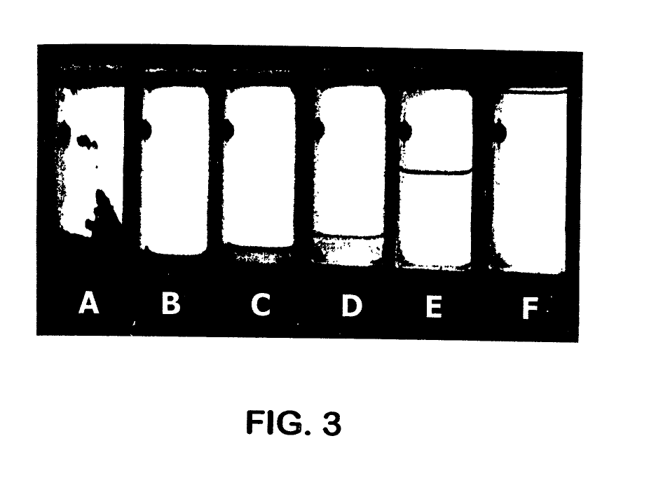

The interaction of BGLU in carbon dioxide was examined in a high-pressure view cell. The BGLU was exposed to carbon dioxide at near room temperature and a pressure of from 35 to 40 bar. The white solid BGLU appeared as a salt as shown in FIG. 3, Panel (A).

### Laboratory Example 2

### Solubility Behavior of β 3 1,2,3,4, 6-Pentaacetyl β-D-Glucose

The procedure according to Example 1 was repeated except that the carbon dioxide pressure was 55.9 bar. A solid to liquid transition (i.e., deliquescence) of the BGLU was observed. See FIG. 3, Panel (B). The melt was observed to absorb carbon dioxide and swell to many times its original volume with gaseous pressure of merely 2 or 3 bar. See FIG. 3, Panel (C) and FIG. 3, Panel (D). Upon reaching liquid-vapor equilibrium pressure, the liquid carbon dioxide formed a separate layer on top of the viscous melt containing carbon dioxide. See FIG. 3, Panel (E). Further addition of carbon dioxide was observed to dilute the liquid phase in this instance. See FIG. 3, Panel (F).

Laboratory Example 3

### Solubility Behavior of β 1,2,3,4, 6-Pentaacetyl β-D-Glucose and α 1,2,3,4, 6-Pentaacetyl α-D-Glucose (AGLU) and 1,2,3,4, 6-Pentaacetyl β-D-Galactose

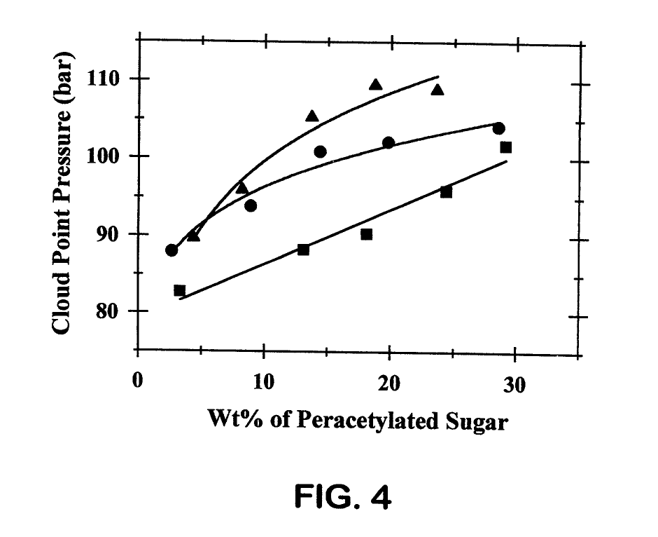

The solubility of BGLU and AGLU in supercritical carbon dioxide were examined at 40° C. It was observed that the solid materials melted, swelled, and readily dissolved in the carbon dioxide. The behavior is illustrated in FIG. 3. At the cloud point pressure, phase separation commences between the supercritical carbon dioxide and the sugar ester melt. Upon lowering the pressure, the material reappears in the solid state (see FIG. 4).

### Results and Discussion of Laboratory Examples 1-3

BGLU is a white solid that melts at 132° C. under atmospheric pressure conditions (FIG. 3, Panel (A)). However, as BGLU is exposed to CO₂ near room temperature (23.0° C.) in a conventional high-pressure view cell, it absorbs CO₂ and becomes “wetted” with CO₂ at a pressure of 35-40 bar. The white solid appears as a salt does in a humid environment. Furthermore, at a gaseous CO₂ pressure of 55.9 bar a solid-to-liquid transition (deliquescence) occurs (FIG. 3, Panel (B)). This is analogous to the deliquescence of hygroscopic materials absorbing atmospheric moisture. The carbohydrate melt continues to absorb CO₂ and swells to many times its original volume with changes in the gaseous CO₂ pressure of only 2 and 3 bar as illustrated in FIG. 3, Panels (C) and (D), respectively. Upon reaching the liquid-vapor equilibrium pressure, the liquid CO₂ forms a separate layer on top of the viscous melt containing CO₂. However, the melt easily mixes with the upper layer of liquid CO₂ on stirring and forms a single-phase liquid mixture in contact with the gaseous CO₂ phase (FIG. 3, Panel (E)). Further addition of CO₂ only dilutes this liquid phase (FIG. 3F). Although, CO₂-induced swelling (Rover et al., (1999) Macromolecules 32: 8965-8973) and CO₂-assisted melting point depression (Zhang & Handa, (1997) Macromolecules 30: 8505-8507.) have been reported in polymers by sorption of CO₂ under high pressures, the materials are not readily miscible in liquid and supercritical CO₂, indicating the lack of a significant attractive interaction.

The deliquescence of BGLU on CO₂ sorption and their mutual miscibility reveal a strong affinity between CO₂ and BGLU, indicating a unique solute-solvent interaction cross-section assisting the formation of solvation shells around the solute molecule.

An approximate estimate of the BGLU concentration in the melt reveals that the system contains more than 80 wt % of BGLU and can be diluted with liquid or scCO₂ in any proportion desired. This indicates that this system, and larger derivatives thereof, can be used for tuning the viscosity of liquid and supercritical CO₂ solutions as desired at low pressures and elevated temperatures. The deliquescence point of AGLU is lower than that of BGLU by about 6-7 bar. BGAL does not exhibit deliquescence though it is readily soluble in liquid CO₂. These observations can be directly correlated to the differences in lattice energies as reflected in the melting points of AGLU, BGLU, and BGAL (109, 132, and 142° C., respectively). Density functional calculations indicate a large number of intramolecular C—H O interactions (FIG. 1) that can play a crucial role in determining the lattice energy by lessening inter-molecular contacts. This can also effectively reduce the CO₂-specific interaction cross-section, which can be reflected in the solubility of the three carbohydrates.

The cloud-point pressures of these systems in scCO₂ were examined at 40.0° C. As in the subcritical case, initially the solid melts and swells (for AGLU and BGLU) and all three peracetylated sugars readily go into a single-phase, scCO₂ system. A plot of the cloud-point pressure versus the weight percent for AGLU, BGLU, and BGAL dissolved in supercritical CO₂ at 40.0° C. is given in FIG. 4. At the cloud-point pressure, phase separation begins between scCO₂ and the sugar ester. Upon lowering the pressure, the material reappears in the solid state. No cloud-point measurements were made above 30% (wt) due to limitations arising from the volume of the view cell and the rapid swelling of the sample in the cases of AGLU and BGLU. Considering this cloud-point data and the data presented in FIG. 3, it is apparent that the mixtures of AGLU and BGLU show complete miscibility at relatively low pressures with the 3-phase line being shifted to extremely low pressures. An understanding of the stereochemical aspects revealed here provides guidance in the design of larger CO₂-philic molecules, since there is a dependence on the configuration of the individual isomers.

### Laboratory Example 4.

Preparation of Glassy Fibers of β-Cyclodextrin Triacetate from a CO₂-induced Melt.

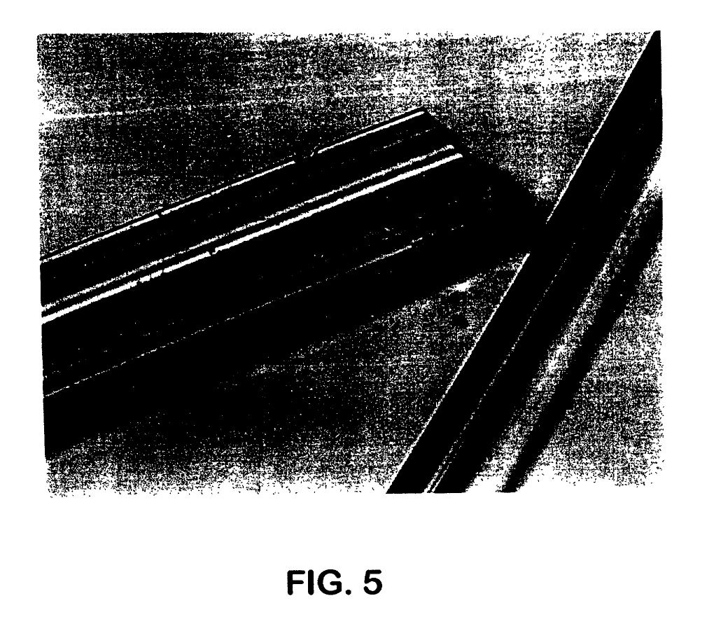

Glassy fibers are prepared from a CO₂-induced melt of β-cyclodextrin triacetate. Initially, the β-cyclodextrin triacetate sample was taken inside a pressure vessel, which was pressurized with CO₂. Once the sample was melted, CO₂ was released. The sample remained liquefied for some time. During this time, a thin glass glass fiber was inserted into the liquefied sample and, when removed, pulled out glassy fibers, as shown in FIG. 5. Fibers of varying lengths (e.g centimeter-length fibers) were pulled from the vessel. The fibers became brittle after the CO₂ escaped completely from the vessel.

### Laboratory Example 5

### Crystallization of BGAL from CO₂

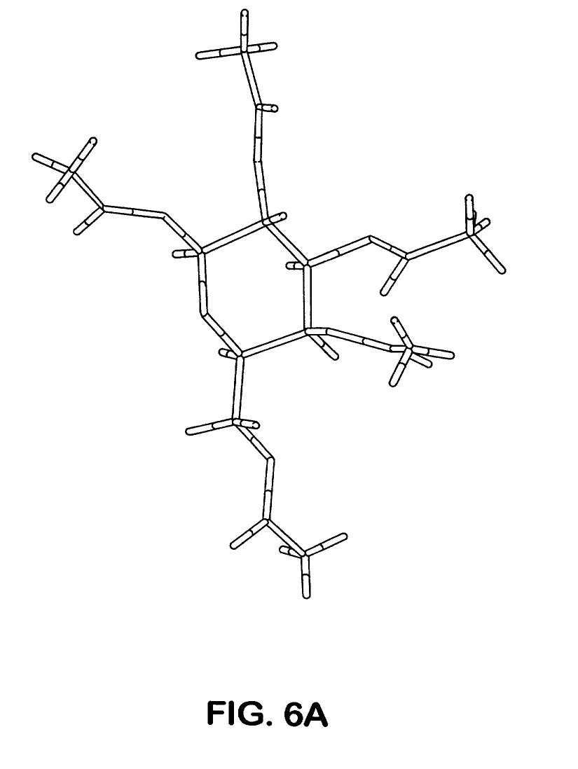

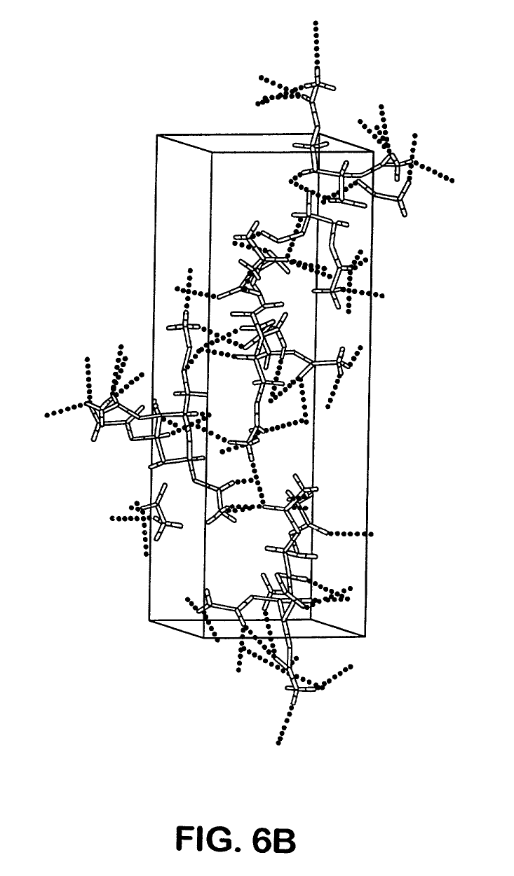

BGAL is crystallized from supercritical carbon dioxide. Approximately 1 gram of BGAL was dissolved in a 9.5 ml volume high pressure cell, which was subsequently pressurized with CO₂ up to 1200 psi pressure at 25° C. The temperature of the cell was raised to 40° C. to maintain supercritical conditions. CO₂ was slowly released through a capillary restrictor overnight and fine crystals of BGAL were obtained. The crystal structure of BGAL was determined using X-ray diffraction techniques and is presented in FIG. 6. The structure of BGAL in the crystal is shown in FIG. 6A while FIG. 6B shows the packing of BGAL molecules inside the crystal.

### References

The references listed below as well as all references cited in the specification are incorporated herein by reference to the extent that they supplement, explain, provide a background for or teach methodology, techniques and/or compositions employed herein.

Consani & Smith, (1990) J. Supercrit. Fluids 3:51-65

DeSimone et al., (1992) Science 267:945-947

Eckert et al., (1996) Nature 373:313-318

Hyatt, (1984) J. Org. Chem. 49: 5097-5101

Johnston et al., (1996) Science 271: 624-626

Kazarian et al., (1996) J. Am. Chem. Soc. 118:1729-1736

Laintz et al., (1991) J. Supercrit. Fluids 4: 194-198

McHugh & Krukonis, (1994) Supercritical Fluid Extractions: Principles and Practice, 2^{nd} ed. Butterworth-Heinerman: Boston, Mass.

Nelson & Borkman, (1998) J. Phys. Chem. A 102:7860-7863

Rindfleisch et al., (1996) J. Phys. Chem. 100:15581-15587

Sarbu et al., (2000) Nature 405:165-168

U.S. Pat. No. 5,733,964

U.S. Pat. No. 5,863,298

U.S. Pat. No. 6,187,911 WO 2001021616

It will be understood that various details of the invention can be changed without departing from the scope of the invention. Furthermore, the foregoing description is for the purpose of illustration only, and not for the purpose of limitation—the invention being defined by the claims.

What is claimed is:

1. A composition comprising a carbohydrate-based material dispersed in carbon dioxide, wherein the carbohydrate-based material comprises a carbohydrate and a non-fluorous CO₂-philic group.

2. The composition of claim 1, wherein the carbon dioxide is in a form selected from the group consisting of supercritical carbon dioxide, liquid carbon dioxide and gaseous carbon dioxide.

3. The composition of claim 1, wherein the carbohydrate is selected from the group consisting of a monosaccharide, a disaccharide, a trisaccharide, a polysaccharide, a cyclic saccharide and an acyclic saccharide.

4. The composition of claim 1, wherein the CO₂-philic group comprises a Lewis base.

5. The composition of claim 1, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

6. A method of forming a composition comprising a carbohydrate-based material dispersed in carbon dioxide, the method comprising:
(a) providing a CO₂-phobic carbohydrate comprising one of one or more hydroxyl groups and one or more or ring hydrogens;
(b) chemically replacing at least one of a hydroxyl group and a ring hydrogen with a non-fluorous CO₂-philic group to form a carbohydrate-based material; and
(c) dispersing the carbohydrate-based material in carbon dioxide, whereby a composition comprising a carbohydrate-based material dispersed in carbon dioxide is formed.

7. The method of claim 6, wherein the CO₂-phobic carbohydrate comprises a moiety selected from the group consisting of alkyl chain, H, a carboxylic acid group, a hydroxyl group, a phosphate group, a phosphate ester group, a sulfonyl group, a sulfate group, a sulfonate group, a branched or straight chained polyalkylene oxide group, an amine oxide group, an alkyl ammonium group, an unsubstituted aryl group substituted, a substituted aryl group, an alkenyl group, a nitryl group, a carbohydrate, H⁺, Na⁺, Li⁺, Ca²⁺, Mg²⁺, Cl⁻, Br⁻, I⁻, a mesylate and a tosylate.

8. The method of claim 6, wherein the carbohydrate is selected from the group consisting of monosaccharides, disaccharides, trisaccharides and polysaccharides.

9. The method of claim 6, wherein the carbohydrate is selected from the group consisting of a cyclic saccharide and an acyclic

10. The method of claim 6, wherein the carbohydrate is an acyclic saccharide.

11. The method of claim 6, wherein the CO₂-philic group is selected from the group consisting of an acetyl group, a benzoyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

12. The method of claim 6, wherein the CO₂-philic group comprises a Lewis base.

13. The method of claim 6, wherein the carbon dioxide is in a form selected from the group consisting of supercritical carbon dioxide, liquid carbon dioxide and gaseous carbon dioxide.

14. A method of modulating the viscosity of a composition comprising carbon dioxide, the method comprising:
(a) providing a carbohydrate-based material adapted for dispersion in carbon dioxide, wherein the carbohydrate-based material comprises a carbohydrate and at least one non-fluorous CO₂-philic group; and
(b) dispersing an amount of the carbohydrate-based material in a composition comprising carbon dioxide sufficient to modulate the viscosity of the composition comprising carbon dioxide to a desired viscosity.

15. The method of claim 14, wherein the carbon dioxide is in a form selected from the group consisting of supercritical carbon dioxide, liquid carbon dioxide and gaseous carbon dioxide.

16. The method of claim 14, wherein the carbohydrate is selected from the group consisting of a monosaccharide, a disaccharide, a trisaccharide, a polysaccharide, a cyclic saccharide and an acyclic saccharide.

17. The method of claim 14, wherein the CO₂-philic group comprises a Lewis base.

18. The method of claim 14, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

19. A method of chelating a metal atom disposed in carbon dioxide, the method comprising:
(a) providing a CO₂-philic carbohydrate-based material comprising a carbohydrate, at least one non-fluorous CO₂-philic group and at least one chelating group covalently linked to one of the CO₂-philic group and the carbohydrate; and
(b) contacting the carbohydrate-based material with a sample comprising carbon dioxide, in which a metal atom is known or suspected to be disposed.

20. The method of claim 19, wherein the carbohydrate is selected from the group consisting of a monosaccharide, a disaccharide, a trisaccharide, a polysaccharide, a cyclic saccharide and an acyclic saccharide.

21. The method of claim 19, wherein the CO₂-philic group comprises a Lewis base.

22. The method of claim 19, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

23. The method of claim 19, wherein the carbon dioxide is in a form selected from the group consisting of supercritical carbon dioxide, liquid carbon dioxide and gaseous carbon dioxide.

24. The method of claim 19, wherein the chelating group is selected from the group consisting of an acetyl acetonate group, a polyaminocarboxylic acid group, a thiocarbamate group, a dithiocarbamate group, a thiol group, an amino group, a picolyl amine group, a bis (picolyl amine) group and a phosphate group.

25. A method of sizing a substrate, the method comprising:
(a) providing a carbohydrate-based material comprising a carbohydrate, at least one non-fluorous CO₂-philic group and at least one moiety known or suspected to be an effective size;
(b) dispersing the carbohydrate-based material in carbon dioxide to form a sizing solution; and
(c) contacting substrate with the sizing solution, whereby a substrate is sized.

26. The method of claim 25, wherein the carbon dioxide is in a form selected from the group consisting of supercritical carbon dioxide, liquid carbon dioxide and gaseous carbon dioxide.

27. The method of claim 25, wherein the carbohydrate is selected from the group consisting of a monosaccharide, a disaccharide, a trisaccharide, a polysaccharide, a cyclic saccharide and an acyclic saccharide.

28. The method of claim 25, wherein the CO₂-philic group comprises a Lewis base.

29. The method of claim 25, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

30. The method of claim 25, wherein the size is selected from the group consisting of an acetylated carbohydrate and a benzoylated carbohydrate.

31. The method of claim 25, wherein the substrate is selected from the group consisting of yarn, paper, a cellulosic material, a non-cellulosic material and wood.

32. A method of sorbing carbon dioxide from a sample, the method comprising:
(a) providing a CO₂-philic carbohydrate-based material comprising a carbohydrate and at least one non-fluorous CO₂-philic group;
(b) contacting the CO₂-philic carbohydrate-based material with a sample known or suspected to comprise carbon dioxide, whereby carbon dioxide is sorbed from a sample.

33. The method of claim 32, wherein the carbohydrate is selected from the group consisting of a monosaccharide, a disaccharide, a trisaccharide, a polysaccharide, a cyclic saccharide and an acyclic saccharide.

34. The method of claim 32, wherein the CO₂-philic group comprises a Lewis base.

35. The method of claim 32, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

36. The method of claim 32, wherein the carbon dioxide is in a form selected from the group consisting of supercritical carbon dioxide, liquid carbon dioxide and gaseous carbon dioxide.

37. The method of claim 32, wherein the sample comprises a byproduct of a combustion event.

38. The method of claim 32, wherein the sample comprises one of a gas emitted from a gas purification system and a gas to be supplied to a gas purification system.

39. A method of isolating a carbohydrate ester from a sample, the method comprising:
(a) providing a sample known or suspected to comprise a carbohydrate ester;
(b) contacting the sample with carbon dioxide to form an extraction mixture; and
(c) isolating the extraction mixture from the sample, whereby a carbohydrate ester is isolated from a sample.

40. The method of claim 39, wherein the carbon dioxide is in a form selected from the group consisting of supercritical carbon dioxide, liquid carbon dioxide and gaseous carbon dioxide.

41. The method of claim 39, wherein the carbohydrate ester comprises a carbohydrate selected from the group consisting of a monosaccharide, a disaccharide, a trisaccharide, a polysaccharide, a cyclic saccharide and an acyclic saccharide.

42. A method of synthesizing a polymer, the method comprising:
(a) providing a carbohydrate-based material comprising a non-fluorous CO₂-philic group;
(b) joining the carbohydrate-based material with a compound comprising a polymerizable group to form a seed unit;
(c) dispersing the seed unit in carbon dioxide; and
(d) initiating polymerization, whereby a polymer is synthesized.

43. The composition of claim 42, wherein the two or more carbohydrate units are selected from the group consisting of a monosaccharide, a disaccharide, a trisaccharide, a polysaccharide, a cyclic saccharide and an acyclic saccharide.

44. The composition of claim 42, wherein the carbon dioxide is in a form selected from the group consisting of supercritical carbon dioxide, liquid carbon dioxide and gaseous carbon dioxide.

45. The method of claim 42, wherein the one or more polymerizable units are selected from the group consisting of ethylene, vinyl acetate, isoprene, allyl substituted compounds and organic compounds comprising a polymerizable double bond.

46. A method of impregnating or plasticizing a matrix comprising a cellulosic or non-cellulosic material, the method comprising:
(a) providing a carbohydrate-based material comprising a carbohydrate, at least one non-fluorous CO₂-philic group and at least one moiety known or suspected to be an effective size;
(b) dispersing the carbohydrate-based material in CO₂ to form a treatment solution;and
(c) contacting a substrate to be impregnated or plasticized with the treatment solution,whereby a matrix comprising a cellulosic or non-cellulosic material is impregnated or plasticized.

47. The method of claim 46, wherein the carbon dioxide is in a form selected from the group consisting of supercritical carbon dioxide, liquid carbon dioxide and gaseous carbon dioxide.

48. The method of claim 46, wherein the carbohydrate is selected from the group consisting of a monosaccharide, a disaccharide, a trisaccharide, a polysaccharide, a cyclic saccharide and an acyclic saccharide.

49. The method of claim 46, wherein the CO₂-philic group comprises a Lewis base.

50. The method of claim 46, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rand R_{n′} and independently hydrogen or an alkyl group.

51. The method of claim 46, wherein the carbohydrate-based material is selected from the group consisting of acetylated carbohydrates and benzoylated carbohydrates.

52. The method of claim 46, wherein the substate can be selected from a cellulosic material such as wood or paper or a non-cellulosic material.

53. A method of isolating a carbohydrate material from a CO₂ solution, the method comprising:
(a) providing a carbohydrate-based material comprising a carbohydrate and a non-fluorous CO₂-philic group;
(b) dispersing the carbohydrate-based material in CO₂ to form a CO₂ solution; and
(c) spraying the CO₂ solution through a nozzle.

54. The method of claim 53, wherein the carbon dioxide is in a form selected from the group consisting of supercritical carbon dioxide, liquid carbon dioxide and gaseous carbon dioxide.

55. The method of claim 53, wherein the carbohydrate is selected from the group consisting of a monosaccharide, a disaccharide, a trisaccharide, a polysaccharide, a cyclic saccharide and an acyclic saccharide.

56. The method of claim 53, wherein the CO₂-philic group comprises a Lewis base.

57. The method of claim 53, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

58. The method of claim 53, wherein the carbohydrate-based material is selected from the group consisting of acetylated carbohydrates and benzoylated carbohydrates.

59. A method of encapsulating a compound in a carbohydrate-based material, the method comprising:
(a) providing a carbohydrate-based material;
(b) dispersing the carbohydrate-based material in CO₂ to form a CO₂ solution; and
(c) dispersing the compound in the CO₂-solution, whereby a compound is encapsulated in a carbohydrate-based material.

60. The method of claim 59, wherein the carbon dioxide is in a form selected from the group consisting of supercritical carbon dioxide, liquid carbon dioxide and gaseous carbon dioxide.

61. The method of claim 59, wherein the carbohydrate is selected from the group consisting of a monosaccharide, a disaccharide, a trisaccharide, a polysaccharide, a cyclic saccharide and an acyclic saccharide.

62. The method of claim 59, wherein the CO₂-philic group comprises a Lewis base.

63. The method of claim 59, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

64. The method of claim 59, wherein the carbohydarte-based material is selected from the group consisting of acetylated carbohydrates and benzoylated carbohydrates.

65. The method of claim 59, wherein the compound is selected from the group consisting of drug molecules and biological molecules.

66. The method of claim 59, wherein the compound is a photographic material.

67. A method of producing a carbohydrate-based mesoporous material, the method comprising:
(a) providing a carbohydrate-based material comprising a carbohydrate and a non-fluorous CO₂-philic group;
(b) dispersing the carbohydrate-based material in CO₂ disposed in a pressurizable vessel to form a CO₂ solution; and
(c) rapidly releasing the CO₂ solution from the vessel, whereby a carbohydrate-based mesoporous material is produced.

68. The method of claim 67, wherein the carbon dioxide is in a form selected from the group consisting of supercritical carbon dioxide, liquid carbon dioxide and gaseous carbon dioxide.

69. The method of claim 67, wherein the carbohydrate is selected from the group consisting of a monosaccharide, a disaccharide, a trisaccharide, a polysaccharide, a cyclic saccharide and an acyclic saccharide.

70. The method of claim 67, wherein the CO₂-philic group comprises a Lewis base.

71. The method of claim 66, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group.

72. The method of claim 67, wherein the carbohydrate-based material is selected from the group consisting of acetylated carbohydrates and benzoylated carbohydrates.

73. A method of crystallizing a carbohydrate-based material from a CO₂ solution, the method comprising:
(a) dispersing a carbohydrate-based material comprising a carbohydrate and a non-fluorous CO₂-philic group in a pressurizable vessel containing CO₂ to form a CO₂ solution; and
(b) expanding the CO₂ solution by slow release of CO₂ from the vessel, whereby a carbohydrate-based material is crystallized.

74. The method of claim 73, wherein the carbon dioxide is in a form selected from the group consisting of supercritical carbon dioxide, liquid carbon dioxide and gaseous carbon dioxide.

75. The method of claim 73, wherein the carbohydrate is selected from the group consisting of a monosaccharide, a disaccharide, a trisaccharide, a polysaccharide, a cyclic saccharide and an acyclic saccharide.

76. The method of claim 73, wherein the CO₂-philic group comprises a Lewis base.

77. The method of claim 73, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} and independently hydrogen or an alkyl group.

78. The method of claim 73, wherein the carbohydrate-based material is selected from the group consisting of acetylated carbohydrates and benzoylated carbohydrates.

79. A method of producing a glassy and fibrous material from a carbohydrate-based material, the method comprising:
(a) melting a carbohydrate-based material comprising a carbohydrate and a non-fluorous CO₂-philic group with CO₂ to form a CO₂ melt;
(b) contacting a crystal formation structure with the CO₂ melt; and
(c) removing the crystal formation structure from the CO₂-melt, whereby a glassy and fibrous material is produced from a carbohydrate-based material.

80. The method of claim 79, wherein the carbon dioxide is in a form selected from the group consisting of supercritical carbon dioxide, liquid carbon dioxide and gaseous carbon dioxide.

81. The method of claim 79, wherein the carbohydrate is selected from the group consisting of a monosaccharide, a disaccharide, a trisaccharide, a polysaccharide, a cyclic saccharide and an acyclic saccharide.

82. The method of claim 79, wherein the CO₂-philic group comprises a Lewis base.

83. The method of claim 79, wherein the CO₂-philic group is selected from the group consisting of an acetyl group, a phosphonyl group, a sulfonyl group, —O—C(O)—Rₙ, —C(O)—Rₙ, —O—P(O)—(O—Rₙ)₂, and —NRₙR_{n′} where Rₙ and R_{n′} are independently hydrogen or an alkyl group

84. The method of claim 79, wherein the CO₂-philic material is selected from the group consisting of acetylated carbohydrates and benzoylated carbohydrates.

85. A method of solubilizing a dye in carbon dioxide, the method comprising:
(a) providing a carbohydrate-based material comprising a carbohydrate and a non-fluorous CO₂-philic group, and a CO₂-phobic dye molecule;
(b) chemically associating the carbohydrate-based material with the CO₂-phobic dye molecule to form a CO₂-soluble dye molecule; and
(c) dispersing the CO₂-soluble dye molecule in CO₂, whereby a dye is solubilized in carbon dioxide.

86. The method of claim 85, wherein the carbon dioxide is in a form selected from the group consisting of supercritical carbon dioxide, liquid carbon dioxide and gaseous carbon dioxide.

87. The method of claim 85, wherein the carbohydrate is selected from the group consisting of a monosaccharide, a disaccharide, a trisaccharide, a polysaccharide, a cyclic saccharide and an acyclic saccharide.

88. The method of claim 85, wherein the CO₂-philic group comprises a Lewis base.

89. The method of claim 85, wherein the carbohydrate-based material is selected from the group consisting of acetylated carbohydrates and benzoylated carbohydrates.

90. A method of solubilizing a catalyst in CO₂, the method comprising:
(a) providing a carbohydrate-based material comprising a carbohydrate and a non-fluorous CO₂-philic group and a catalyst molecule;
(b) chemically associating the carbohydrate-based material and the catalyst molecule to form a CO₂ soluble catalyst; and
(c) dispersing the CO₂ soluble catalyst in CO₂, whereby a catalyst is solubilized in CO₂.

91. The method of claim 90, wherein the carbon dioxide is in a form selected from the group consisting of supercritical carbon dioxide, liquid carbon dioxide and gaseous carbon dioxide.

92. The method of claim 90, wherein the carbohydrate is selected from the group consisting of a monosaccharide, a disaccharide, a trisaccharide, a polysaccharide, a cyclic saccharide and an acyclic saccharide.

93. The method of claim 90, wherein the CO₂-philic group comprises a Lewis base.

94. The method of claim 90, wherein the carbohydrate-based material is selected from the group consisting of acetylated carbohydrates and benzoylated carbohydrates.

95. A method of extracting a carbohydrate-containing molecule from a matrix using CO₂, the method comprising:
(a) providing a matrix comprising a CO₂-phobic carbohydrate-containing molecule;
(b) contacting the matrix with acetic anhydride and acetic acid to form an acetylated carbohydrate-containing molecule;
(c) extracting the acetylated carbohydrate molecule from the matrix, using carbon dioxide as a solvent to form extracted carbohydrate molecules; and
(d) hydrolyzing the extracted carbohydrate molecules, whereby a carbohydrate-containing molecule is extracted.

96. The method of claim 95, wherein the carbon dioxide is in a form selected from the group consisting of supercritical carbon dioxide, liquid carbon dioxide and gaseous carbon dioxide.

97. The method of claim 95, wherein the carbohydrate-containing molecule is selected from the group consisting of a monosaccharide, a disaccharide, a trisaccharide, a polysaccharide, a cyclic saccharide and an acyclic saccharide.
